(19) United States
(12) Patent Application Publication
(10) Pub. No.: US 2020/0330557 A1
(43) Pub. Date: Oct. 22, 2020

(54) USES OF PD-1/PD-L1 INHIBITORS AND/OR CTLA-4 INHIBITORS WITH A BIOLOGIC CONTAINING MULTIPLE CYTOKINE COMPONENTS TO TREAT CANCER

(71) Applicant: Brooklyn Immunotherapeutics LLC, New York, NY (US)

(72) Inventors: John W. Hadden II, New York, NY (US); Neil L. Berinstein, Toronto (CA); James E. Egan, Stony Brook, NY (US)

(73) Assignee: Brooklyn Immunotherapeutics LLC, New York, NY (US)

(21) Appl. No.: 16/326,611

(22) PCT Filed: Aug. 18, 2017
(86) PCT No.: PCT/US2017/047477

§ 371 Date: Feb. 19, 2019

Related U.S. Application Data
(60) Provisional application No. 62/377,051, filed on Aug. 19, 2016.

(51) Int. Cl. A61K 38/20; A61K 38/19; A61K 38/21; C07K 16/28; A61K 31/675; A61K 31/404; A61K 33/30; G01N 33/574; A61P 35/00

(52) U.S. Cl. CPC A61K 38/2006; A61K 38/2013; A61K 38/204; A61K 38/2053; A61K 38/191; A61K 38/217; A61K 9/0019; C07K 16/2827; A61K 31/675; A61K 31/404; A61K 33/30; G01N 33/57484; A61P 35/00; C07K 16/2818

(57) ABSTRACT

Aspects of the disclosure relate to methods for treating cancer, e.g., by administering to a subject having cancer a primary cell-derived biologic with multiple cytokine components in combination with an antagonist of programmed cell death-ligand 1 (PD-L1) or programmed cell death 1 (PD-1) and/or with an antagonist of cytotoxic T-lymphocyte-associated protein 4 (CTLA-4). Other aspects of the disclosure relate to methods of identifying a subject for treatment with an antagonist of PD-L1 or PD-1 and/or an antagonist of CTLA-4 or assessing the likelihood that a subject will be responsive to an antagonist of PD-L1 or PD-1 and/or an antagonist of CTLA-4.

### CROSS-REFERENCE TO RELATED APPLICATIONS

This application claims the benefit under 35 U.S.C. § 119(e) of U.S. Provisional Application No. US 62/377,051 filed on Aug. 19, 2016, the contents of which are incorporated herein by reference in its entirety.

### BACKGROUND OF INVENTION

PD-1 and PD-L1 inhibitors are checkpoint inhibitors that are used to treat various forms of cancer. Unfortunately, PD-L1 expression in the tumor microenvironment, which may be one of the important parameters that correlates with and may even be required for efficacy of PD-1/PD-L1 inhibitors, varies by tumor type and among individual patients (see, e.g., Taube et al., Clin Cancer Res; 20(19):5064-74 (2014) and Sunshine and Taube, Current Opinion in Pharmacology, 23:32-38 (2015)). CTLA-4 inhibitors are also checkpoint inhibitors that are being developed to treat various forms of cancer. CTLA-4 expression has also been shown to correlate with efficacy of CTLA-4 inhibitors. As a result, there remains a need to increase the fraction of patients who benefit from treatment with PD-1/PD-L1 and/or CTLA-4 inhibitors, or to enhance the magnitude of response to such inhibitors.

### SUMMARY OF THE INVENTION

Aspects of the disclosure relate to methods and compositions that utilize a primary cell-derived biologic to enhance the therapeutic efficacy of antagonists of programmed cell death-ligand 1 (PD-L1), programmed cell death 1 (PD-1) and/or cytotoxic T-lymphocyte-associated protein 4 (CTLA-4), such as for treatment of cancer. As described herein, it has been surprisingly found that distal administration of a primary cell-derived biologic comprising IL-1β, IL-2, IL-6, IL-8, TNF-α, and IFN-γ to the lymph nodes of cancer patients results in strong local upregulation of PD-L1 and CTLA-4 within the tumor. PD-L1 expression on tumor cells, on infiltrating immune cells, and in the tumor microenvironment is strongly correlated with and may be required for PD-1/PD-L1 antagonist efficacy (see, e.g., Taube et al., Clin Cancer Res; 20(19):5064-74 (2014); Sunshine and Taube, Current Opinion in Pharmacology, 23:32-38 (2015); Garon et al. N Engl J Med, 372:2018-2028 (2015); Schmid et al. European Cancer Congress, Abstract Number 3017 (2015); and Carbognin et al. PLoS ONE 10(6): e0130142. (2015)). Increased CTLA-4 expression has also been shown to correlate with increased CTLA-4 antagonist efficacy (see, e.g., Jamieson et al. Gene-expression profiling to predict responsiveness to immunotherapy. Cancer Gene Therapy (2017) 24:134-140 and Van Allen et al. Genomic correlates of response to CTLA-4 blockade in metastatic melanoma. Science (2015) 350(6257):207-211). Accordingly, without wishing to be bound by theory, it is expected that administration of a primary cell-derived biologic, which as demonstrated herein results in upregulation of PD-L1 by the tumor itself or immune cells infiltrating the tumor, will increase the efficacy of PD-L1 or PD-1 antagonists, e.g., by increasing the number of patients who respond to the antagonists and/or by making the antagonistic response more robust. Similarly, again without wishing to be bound by theory, it is expected that administration of a primary cell-derived biologic, which as demonstrated herein results in upregulation of CTLA-4 within the tumor microenvironment (presumably on immune cells infiltrating the tumor), will increase the efficacy of CTLA-4 antagonists, e.g., by increasing the number of patients who respond to the antagonists and/or by making the antagonistic response more robust.

In some aspects, the disclosure provides a method of treating cancer or a pre-cancerous lesion in a subject (e.g., a human subject), the method comprising (a) administering to a subject having cancer or a pre-cancerous lesion an effective amount of a primary cell-derived biologic comprising IL-1β, IL-2, IL-6, IL-8, TNF-α, and IFN-γ (e.g., human IL-1β, human IL-2, human IL-6, human IL-8, human TNF-α, and human IFN-γ); and (b) administering to the subject an effective amount of an antagonist of programmed cell death-ligand 1 (PD-L1) or programmed cell death 1 (PD-1), wherein the administration of the primary cell-derived biologic and the administration of the antagonist occur at different locations in the subject and/or at different times.

In some embodiments, at least one administration of the primary cell-derived biologic occurs prior to at least one administration of the antagonist. In some embodiments, at least one administration of the primary cell-derived biologic occurs prior to at least one administration of the antagonist and at least one further administration of the primary cell-derived biologic occurs after the at least one administration of the antagonist. In some embodiments, at least one administration of the antagonist occurs prior to at least one administration of the primary cell-derived biologic. In some embodiments, at least one administration of the antagonist occurs prior to at least one administration of the primary cell-derived biologic and at least one further administration of the antagonist occurs after the at least one administration of the primary cell-derived biologic.

In some embodiments, the primary cell-derived biologic is administered subcutaneously or perilymphatically and the antagonist is administered intravenously or orally. In some embodiments, the primary cell-derived biologic is administered once a day for 10 days and the antagonist of PD-L1 or PD-1 is administered once every two to four weeks.

In some embodiments, the antagonist is an antisense oligonucleotide, a short interfering RNA (siRNA), small molecule, a peptide, or an antibody. In some embodiments, the antagonist is an antibody. In some embodiments, the antibody is a human or humanized antibody. In some embodiments, the antibody is specific for PD-L1. In some embodiments, the antibody is selected from the group consisting of atezolizumab, durvalumab, BMS-936559, and avelumab. In some embodiments, the antagonist is CA-170. In some embodiments, the antibody is specific for PD-1. In some embodiments, the antibody is selected from the group consisting of nivolumab, pidilizumab, pembrolizumab, MEDI-0680, and REGN2810. In some embodiments, the antagonist is AMP-224.

In some embodiments, the subject is refractory to treatment with the antagonist prior to administration of the primary cell-derived biologic. In some embodiments, a level of PD-L1 in a tumor of the subject increases after administration of the primary cell-derived biologic.

In some embodiments, the effective amount of the primary cell-derived biologic administered to the subject includes a ratio of IL-1β International Units (IU) to IL-2 IU of 0.45 to 1.37, a ratio of IFN-γ IU to IL-2 IU of 0.19 to 0.39, a ratio of TNF-ααIU to IL-2 IU of 0.53 to 1.26, a ratio of IL-6 IU to IL-2 IU of 1.16 to 6.06, and a ratio of IL-8 IU to IL-2 IU of 0.15 to 0.51. In some embodiments, the effective amount of the primary cell-derived biologic administered to the subject includes at least 1 IU of each of IL-1β, IL-2, IL-6, IL-8, and TNF-α, and IFN-γ. In some embodiments, the effective amount of the primary cell-derived biologic administered to the subject includes at least 2 IU of each of IL-1β, IL-2, IL-6, IL-8, and TNF-α, and IFN-γ. In some embodiments, the effective amount of the primary cell-derived biologic administered to the subject includes at least 3 IU of each of IL-1β, IL-2, IL-6, IL-8, and TNF-α, and IFN-γ. In some embodiments, the effective amount of the primary cell-derived biologic administered to the subject includes 22-657 International Units (IU) of IL-1β, 29-478 IU of IL-2, 10-185 IU of IFN-γ, 29-600 IU of TNF-α, 34-2,895 IU of IL-6 and 5-244 IU of IL-8. IU values may be calculated as described herein. In some embodiments, the effective amount of the primary cell-derived biologic administered to the subject includes 220-6,700 pcg of IL-1β, 1730-28,100 pcg of IL-2, 560-10,900 pcg of IFN-γ, 580-12,000 pcg of TNF-α. 260-22,100 pcg of IL-6, and 4,610-243,600 pcg of IL-8. In some embodiments, the primary cell-derived biologic further comprises GM-CSF and G-CSF.

In some embodiments, the method further comprises administering to the subject a chemical inhibitor selected from the group consisting of alkylating agents, antimetabolites, antibiotics, and immunomodulating agents. In some embodiments, the alkylating agent is cyclophosphamide. In some embodiments, the method further comprises administering to the subject an NSAID selected from the group consisting of indomethacin, ibuprofen, celecoxib, rofecoxib, and combinations thereof. In some embodiments, the NSAID is indomethacin. In some embodiments of any of the methods provided herein, the method further comprises administering zinc to the subject.

In some embodiments, the method further comprises administering to the subject an effective amount of a cytotoxic T-lymphocyte-associated protein 4 (CTLA-4) antagonist. In some embodiments, the CTLA-4 antagonist is an antisense oligonucleotide, a short interfering RNA (siRNA), small molecule, a peptide, or an antibody. In some embodiments, the CTLA-4 antagonist is an antibody (e.g., a human or humanized antibody). In some embodiments, the CTLA-4 antagonist is an antibody selected from the group consisting of ipilimumab and tremelimumab.

In some embodiments, the cancer is selected from the group consisting of melanoma, lung cancer (such as non-small-cell lung cancer (NSCLC) or SCLC), renal cell carcinoma (RCC), prostate cancer, ovarian cancer, colorectal cancer (CRC), kidney cancer, gastric cancer, breast cancer, diffuse large B cell lymphoma (DLBCL), hematological malignancies (e.g., acute myelogenous leukemia (AML), multiple myeloma (MM), chronic lymphocytic leukemia (CLL), Chronic myelogenous leukemia (CML), Hodgkin's lymphoma, non-Hodgkin's lymphoma, B-cell lymphoma, follicular lymphoma, cutaneous T-cell lymphoma), pancreatic cancer, bladder cancer, squamous cell carcinoma of the head and neck (H&NSCC also called SCCHN), genitourinary cancer, advanced cutaneous squamous cell carcinoma, liver metastases, mesothelioma, gastroesophageal cancer, Merkel cell carcinoma, and urothelial carcinoma.

In other aspects, the disclosure provides a method of treating cancer or a pre-cancerous lesion in a subject (e.g., a human subject), the method comprising (a) administering to a subject having cancer or a pre-cancerous lesion an effective amount of a primary cell-derived biologic comprising IL-1β, IL-2, IL-6, IL-8, TNF-α, and IFN-γ (e.g., human IL-1β, human IL-2, human IL-6, human IL-8, human TNF-α, and human IFN-γ); and (b) administering to the subject an effective amount of an antagonist of programmed cell death-ligand 1 (PD-L1) or programmed cell death 1 (PD-1), wherein the antagonist is selected from the group consisting of nivolumab, pidilizumab, pembrolizumab, MEDI-0680, REGN2810, AMP-224, atezolizumab, durvalumab, BMS-936559, avelumab, and CA-170.

In some embodiments, the effective amount of the primary cell-derived biologic administered to the subject includes a ratio of IL-1β International Units (IU) to IL-2 IU of 0.45 to 1.37, a ratio of IFN-γ IU to IL-2 IU of 0.19 to 0.39, a ratio of TNF-α IU to IL-2 IU of 0.53 to 1.26, a ratio of IL-6 IU to IL-2 IU of 1.16 to 6.06, and a ratio of IL-8 IU to IL-2 IU of 0.15 to 0.51. In some embodiments, the effective amount of the primary cell-derived biologic administered to the subject includes at least 1 IU of each of IL-1β, IL-2, IL-6, IL-8, and TNF-α, and IFN-γ. In some embodiments, the effective amount of the primary cell-derived biologic administered to the subject includes at least 2 IU of each of IL-1β, IL-2, IL-6, IL-8, and TNF-α, and IFN-γ. In some embodiments, the effective amount of the primary cell-derived biologic administered to the subject includes at least 3 IU of each of IL-1β, IL-2, IL-6, IL-8, and TNF-α, and IFN-γ. In some embodiments, the effective amount of the primary cell-derived biologic administered to the subject includes 22-657 International Units (IU) of IL-1β, 29-478 IU of IL-2, 10-185 IU of IFN-γ, 29-600 IU of TNF-α, 34-2,895 IU of IL-6 and 5-244 IU of IL-8. In some embodiments, the effective amount of the primary cell-derived biologic administered to the subject includes 220-6,700 pcg of IL-1β, 1730-28,100 pcg of IL-2, 560-10,900 pcg of IFN-γ, 580-12,000 pcg of TNF-α. 260-22,100 pcg of IL-6, and 4,610-243,600 pcg of IL-8. In some embodiments, the primary cell-derived biologic further comprises GM-CSF and G-CSF.

In some embodiments, the method further comprises administering to the subject a chemical inhibitor selected from the group consisting of alkylating agents, antimetabolites, antibiotics, and immunomodulating agents. In some embodiments, the alkylating agent is cyclophosphamide. In some embodiments, the method further comprises administering to the subject an NSAID selected from the group consisting of indomethacin, ibuprofen, celecoxib, rofecoxib, and combinations thereof. In some embodiments, the NSAID is indomethacin. In some embodiments of any of the methods provided herein, the method further comprises administering zinc to the subject.

In some embodiments, the method further comprises administering to the subject an effective amount of a cytotoxic T-lymphocyte-associated protein 4 (CTLA-4) antagonist. In some embodiments, the CTLA-4 antagonist is an antisense oligonucleotide, a short interfering RNA (siRNA), small molecule, a peptide, or an antibody. In some embodiments, the CTLA-4 antagonist is an antibody (e.g., a human or humanized antibody). In some embodiments, the CTLA-4 antagonist is an antibody selected from the group consisting of ipilimumab and tremelimumab.

In some embodiments, the cancer is selected from the group consisting of melanoma, lung cancer (such as non-small-cell lung cancer (NSCLC) or small-cell lung cancer (SCLC)), renal cell carcinoma (RCC), prostate cancer, ovarian cancer, colorectal cancer (CRC), kidney cancer, gastric cancer, breast cancer, diffuse large B cell lymphoma (DLBCL), hematological malignancies (e.g., acute myelogenous leukemia (AML), multiple myeloma (MM), chronic lymphocytic leukemia (CLL), Chronic myelogenous leukemia (CML), Hodgkin's lymphoma, non-Hodgkin's lymphoma, B-cell lymphoma, follicular lymphoma, cutaneous T-cell lymphoma), pancreatic cancer, bladder cancer, squamous cell carcinoma of the head and neck (H&NSCC also called SCCHN), genitourinary cancer, advanced cutaneous squamous cell carcinoma, liver metastases, mesothelioma, gastroesophageal cancer, Merkel cell carcinoma, and urothelial carcinoma.

In yet other aspects, the disclosure provides a method of selecting a subject (e.g., a human subject) for treatment, the method comprising (a) determining a level of PD-L1 in a tumor sample obtained from a subject having cancer or a pre-cancerous lesion and to whom has been administered a primary cell-derived biologic comprising IL-1β, IL-2, IL-6, IL-8, TNF-α, and IFN-γ (e.g., human IL-1β, human IL-2, human IL-6, human IL-8, human TNF-α. and human IFN-γ); and (b) administering to the subject an effective amount of an antagonist of PD-L1 or PD-1 if the level of PD-L1 in the tumor sample is higher than a threshold level of PD-L1.

In some embodiments, determining comprises performing an assay to detect the level of PD-L1. In some embodiments, the assay is selected from the group consisting of in situ hybridization, RT-qPCR, microarray analysis, multiplexed RNA expression analysis, RNA-seq, an immunohistochemistry assay, flow cytometry, a multiplexed protein assay and a Western blot assay. In some embodiments, the level of PD-L1 in the tumor sample is a level of PD-L1 in cell membranes (e.g., tumor cell membranes, immune infiltrate cell membranes, and/or stromal cell membranes) in the tumor sample. In some embodiments, determining comprises performing an immunohistochemistry assay and the threshold level of PD-L1 is partial or complete cell membrane staining in 49% of viable tumor cells in the tumor sample. In some embodiments, the method further comprises administering the primary cell-derived biologic to the subject prior to the determining step.

In some embodiments, the antagonist is an antisense oligonucleotide, a short interfering RNA (siRNA), small molecule, a peptide, or an antibody. In some embodiments, the antagonist is an antibody. In some embodiments, the antibody is a human or humanized antibody. In some embodiments, the antibody is specific for PD-L1. In some embodiments, the antibody is selected from the group consisting of atezolizumab, durvalumab, BMS-936559, and avelumab. In some embodiments, the antagonist is CA-170. In some embodiments, the antibody is specific for PD-1. In some embodiments, the antibody is selected from the group consisting of nivolumab, pidilizumab, pembrolizumab, MEDI-0680, and REGN2810. In some embodiments, the antagonist is AMP-224.

In some embodiments, the effective amount of the primary cell-derived biologic administered to the subject includes a ratio of IL-1β International Units (IU) to IL-2 IU of 0.45 to 1.37, a ratio of IFN-γ IU to IL-2 IU of 0.19 to 0.39, a ratio of TNF-α IU to IL-2 IU of 0.53 to 1.26, a ratio of IL-6 IU to IL-2 IU of 1.16 to 6.06, and a ratio of IL-8 IU to IL-2 IU of 0.15 to 0.51. In some embodiments, the primary cell-derived biologic includes at least 1 IU of each of IL-1β, IL-2, IL-6, IL-8, and TNF-α, and IFN-γ. In some embodiments, the effective amount of the primary cell-derived biologic administered to the subject includes at least 2 IU of each of IL-1β, IL-2, IL-6, IL-8, and TNF-α, and IFN-γ. In some embodiments, the effective amount of the primary cell-derived biologic administered to the subject includes at least 3 IU of each of IL-1β, IL-2, IL-6, IL-8, and TNF-α, and IFN-γ. In some embodiments, the primary cell-derived biologic includes 22-657 International Units (IU) of IL-1(3, 29-478 IU of IL-2, 10-185 IU of IFN-γ, 29-600 IU of TNF-α, 34-2,895 IU of IL-6 and 5-244 IU of IL-8. In some embodiments, the effective amount of the primary cell-derived biologic administered to the subject includes 220-6,700 pcg of IL-1(3, 1730-28,100 pcg of IL-2, 560-10,900 pcg of IFN-γ, 580-12,000 pcg of TNF-α. 260-22,100 pcg of IL-6, and 4,610-243,600 pcg of IL-8. In some embodiments, the primary cell-derived biologic further comprises GM-CSF and G-CSF.

In some embodiments, the cancer is selected from the group consisting of melanoma, lung cancer (such as non-small-cell lung cancer (NSCLC) and SCLC), renal cell carcinoma (RCC), prostate cancer, ovarian cancer, colorectal cancer (CRC), kidney cancer, gastric cancer, breast cancer, diffuse large B cell lymphoma (DLBCL), hematological malignancies (e.g., acute myelogenous leukemia (AML), multiple myeloma (MM), chronic lymphocytic leukemia (CLL), Chronic myelogenous leukemia (CML), Hodgkin's lymphoma, non-Hodgkin's lymphoma, B-cell lymphoma, follicular lymphoma, cutaneous T-cell lymphoma), pancreatic cancer, bladder cancer, squamous cell carcinoma of the head and neck (H&NSCC also called SCCHN), genitourinary cancer, advanced cutaneous squamous cell carcinoma, liver metastases, mesothelioma, gastroesophageal cancer, Merkel cell carcinoma, and urothelial carcinoma.

In other aspects, the disclosure provides a method of assessing the likelihood that a subject (e.g., a human subject) will be responsive to an antagonist of PD-L1 or PD-1, the method comprising (a) administering a primary cell-derived biologic comprising IL-1β, IL-2, IL-6, IL-8, TNF-α, and IFN-γ (e.g., human IL-1β, human IL-2, human IL-6, human IL-8, human TNF-α, and human IFN-γ) to a subject having a cancer or a pre-cancerous lesion that expresses a first level of PD-L1 that is below a threshold level of PD-L1; and (b) determining a second level of PD-L1 in a tumor sample from the subject after administration of the primary cell-derived biologic, wherein a second level of PD-L1 that is above the threshold level of PD-L1 is indicative that the subject will be responsive to the antagonist of PD-L1 or PD-1.

In some embodiments, determining comprises performing an assay to detect the second level of PD-L1. In some embodiments, the assay is selected from the group consisting of in situ hybridization, RT-qPCR, microarray analysis, multiplexed RNA expression analysis, RNA-seq, an immunohistochemistry assay, flow cytometry, a multiplexed protein assay and a Western blot assay. In some embodiments, the second level of PD-L1 is a level of PD-L1 in cell membranes (e.g., tumor cell membranes, immune infiltrate cell membranes, and/or stromal cell membranes) in the tumor sample. In some embodiments, determining comprises performing an immunohistochemistry assay and the threshold level of PD-L1 is partial or complete cell membrane staining of at least 49% of viable tumor cells in the tumor sample.

In some embodiments, the cancer is selected from the group consisting of melanoma, lung cancer (such as non-small-cell lung cancer (NSCLC) or SCLC), renal cell carcinoma (RCC), prostate cancer, ovarian cancer, colorectal cancer (CRC), kidney cancer, gastric cancer, breast cancer, diffuse large B cell lymphoma (DLBCL), hematological malignancies (e.g., acute myelogenous leukemia (AML), multiple myeloma (MM), chronic lymphocytic leukemia (CLL), Chronic myelogenous leukemia (CML), Hodgkin's lymphoma, non-Hodgkin's lymphoma, B-cell lymphoma, follicular lymphoma, cutaneous T-cell lymphoma), pancreatic cancer, bladder cancer, squamous cell carcinoma of the head and neck (H&NSCC also called SCCHN), genitourinary cancer, advanced cutaneous squamous cell carcinoma, liver metastases, mesothelioma, gastroesophageal cancer, Merkel cell carcinoma, and urothelial carcinoma.

In yet other aspects, the disclosure provides a method of treating cancer in a subject (e.g., a human subject), the method comprising: administering to a subject having cancer an effective amount of a primary cell-derived biologic comprising IL-1β, IL-2, IL-6, IL-8, TNF-α, and IFN-γ (e.g., human IL-1β, human IL-2, human IL-6, human IL-8, human TNF-α, and human IFN-γ); and administering to the subject an effective amount of an antagonist of cytotoxic T-lymphocyte-associated protein 4 (CTLA-4), wherein the administration of the primary cell-derived biologic and the administration of the antagonist occur at different locations in the subject and/or at different times.

In some embodiments, at least one administration of the primary cell-derived biologic occurs prior to at least one administration of the antagonist. In some embodiments, at least one administration of the primary cell-derived biologic occurs prior to at least one administration of the antagonist and at least one further administration of the primary cell-derived biologic occurs after the at least one administration of the antagonist. In some embodiments, at least one administration of the antagonist occurs prior to at least one administration of the primary cell-derived biologic. In some embodiments, at least one administration of the antagonist occurs prior to at least one administration of the primary cell-derived biologic and at least one further administration of the antagonist occurs after the at least one administration of the primary cell-derived biologic. In some embodiments, the primary cell-derived biologic is administered subcutaneously or perilymphatically and the antagonist is administered intravenously. In some embodiments, the primary cell-derived biologic is administered once a day up to 10 days and the antagonist is administered once every three to twelve weeks.

In some embodiments, the CTLA-4 antagonist is an antisense oligonucleotide, a short interfering RNA (siRNA), small molecule, a peptide, or an antibody. In some embodiments, the antagonist is an antibody. In some embodiments, the antibody is a human or humanized antibody. In some embodiments, the antibody is selected from the group consisting of ipilimumab and tremelimumab.

In some embodiments, the subject is refractory to treatment with the antagonist prior to administration of the primary cell-derived biologic. In some embodiments, a level of CTLA-4 in a tumor of the subject increases after administration of the primary cell-derived biologic.

In some embodiments, the effective amount of the primary cell-derived biologic administered to the subject includes a ratio of IL-1β International Units (IU) to IL-2 IU of 0.45 to 1.37, a ratio of IFN-γ IU to IL-2 IU of 0.19 to 0.39, a ratio of TNF-α IU to IL-2 IU of 0.53 to 1.26, a ratio of IL-6 IU to IL-2 IU of 1.16 to 6.06, and a ratio of IL-8 IU to IL-2 IU of 0.15 to 0.51. In some embodiments, the effective amount of the primary cell-derived biologic administered to the subject includes at least 1 IU of each of IL-1β, IL-2, IL-6, IL-8, and TNF-α. In some embodiments, the effective amount of the primary cell-derived biologic administered to the subject includes 22-657 International Units (IU) or 220-6,700 pcg of IL-1β, 29-478 IU or 1730-28,100 pcg of IL-2, 10-185 IU or 560-10,900 pcg of IFN-γ, 29-600 IU or 580-12,000 pcg of TNF-α, 34-2,895 IU or 260-22,100 pcg of IL-6 and 5-244 IU or 4,610-243,600 of IL-8. In some embodiments, the primary cell-derived biologic further comprises GM-CSF and G-CSF.

In some embodiments, the method further comprises administering to the subject a chemical inhibitor selected from the group consisting of alkylating agents, antimetabolites, antibiotics, and immunomodulating agents. In some embodiments, the alkylating agent is cyclophosphamide. In some embodiments, the method further comprises administering to the subject an NSAID selected from the group consisting of indomethacin, ibuprofen, celecoxib, rofecoxib, and combinations thereof. In some embodiments, the NSAID is indomethacin. In some embodiments, the method further comprises administering zinc to the subject.

In some embodiments, the method further comprises administering an effective amount of a PD-1 or PD-L1 antagonist. In some embodiments, the PD-1 or PD-L1 antagonist is an antisense oligonucleotide, a short interfering RNA (siRNA), small molecule, a peptide, or an antibody. In some embodiments, the PD-1 or PD-L1 antagonist is an antibody. In some embodiments, the antibody is a human or humanized antibody. In some embodiments, the PD-1 or PD-L1 antagonist is selected from the group consisting of nivolumab, pidilizumab, pembrolizumab, MEDI-0680, REGN2810, AMP-224, atezolizumab, durvalumab, BMS-936559, avelumab, and CA-170.

In some embodiments, the cancer is selected from the group consisting of melanoma, lung cancer (such as non-small-cell lung cancer (NSCLC) or SCLC), renal cell carcinoma (RCC), prostate cancer, ovarian cancer, colorectal cancer (CRC), kidney cancer, gastric cancer, breast cancer, diffuse large B cell lymphoma (DLBCL), hematological malignancies (e.g., acute myelogenous leukemia (AML), multiple myeloma (MM), chronic lymphocytic leukemia (CLL), Chronic myelogenous leukemia (CML), Hodgkin's lymphoma, non-Hodgkin's lymphoma, B-cell lymphoma, follicular lymphoma, cutaneous T-cell lymphoma), pancreatic cancer, bladder cancer, squamous cell carcinoma of the head and neck, genitourinary cancer, advanced cutaneous squamous cell carcinoma, liver metastases, mesothelioma, gastroesophageal cancer, Merkel cell carcinoma, and urothelial carcinoma.

In another aspect, the disclosure provides a method of treating cancer in a subject (e.g., a human subject), the method comprising administering to a subject having cancer an effective amount of a primary cell-derived biologic comprising IL-1β, IL-2, IL-6, IL-8, TNF-α, and IFN-γ (e.g., human IL-1β, human IL-2, human IL-6, human IL-8, human TNF-α, and human IFN-γ); and administering to the subject an effective amount of an antagonist of CTLA-4, wherein the antagonist is selected from the group consisting of ipilimumab and tremelimumab.

In some embodiments, the effective amount of the primary cell-derived biologic administered to the subject includes a ratio of IL-1β International Units (IU) to IL-2 IU of 0.45 to 1.37, a ratio of IFN-γ IU to IL-2 IU of 0.19 to 0.39, a ratio of TNF-α IU to IL-2 IU of 0.53 to 1.26, a ratio of IL-6 IU to IL-2 IU of 1.16 to 6.06, and a ratio of IL-8 IU to IL-2 IU of 0.15 to 0.51. In some embodiments, the effective amount of the primary cell-derived biologic administered to the subject includes at least 1 IU of each of IL-1β, IL-2, IL-6, IL-8, and TNF-α. In some embodiments, the effective amount of the primary cell-derived biologic administered to the subject includes 22-657 International Units (IU) or 220-6,700 pcg of IL-1β, 29-478 IU or 1730-28,100 pcg of IL-2, 10-185 IU or 560-10,900 pcg of IFN-γ, 29-600 IU or 580-12,000 pcg of TNF-α, 34-2,895 IU or 260-22,100 pcg of IL-6 and 5-244 IU or 4,610-243,600 of IL-8. In some embodiments, the primary cell-derived biologic further comprises GM-CSF and G-CSF.

In some embodiments, the method further comprises administering to the subject a chemical inhibitor selected from the group consisting of alkylating agents, antimetabolites, antibiotics, and immunomodulating agents. In some embodiments, the alkylating agent is cyclophosphamide. In some embodiments, the method further comprises administering to the subject an NSAID selected from the group consisting of indomethacin, ibuprofen, celecoxib, rofecoxib, and combinations thereof. In some embodiments, the NSAID is indomethacin. In some embodiments, the method further comprises administering zinc to the subject.

In some embodiments, the method further comprises administering an effective amount of a PD-1 or PD-L1 antagonist. In some embodiments, the PD-1 or PD-L1 antagonist is an antisense oligonucleotide, a short interfering RNA (siRNA), small molecule, a peptide, or an antibody. In some embodiments, the PD-1 or PD-L1 antagonist is an antibody. In some embodiments, the antibody is a human or humanized antibody. In some embodiments, the PD-1 or PD-L1 antagonist is selected from the group consisting of nivolumab, pidilizumab, pembrolizumab, MEDI-0680, REGN2810, AMP-224, atezolizumab, durvalumab, BMS-936559, avelumab, and CA-170.

In some embodiments, the cancer is selected from the group consisting of melanoma, lung cancer (such as non-small-cell lung cancer (NSCLC) or SCLC), renal cell carcinoma (RCC), prostate cancer, ovarian cancer, colorectal cancer (CRC), kidney cancer, gastric cancer, breast cancer, diffuse large B cell lymphoma (DLBCL), hematological malignancies (e.g., acute myelogenous leukemia (AML), multiple myeloma (MM), chronic lymphocytic leukemia (CLL), Chronic myelogenous leukemia (CML), Hodgkin's lymphoma, non-Hodgkin's lymphoma, B-cell lymphoma, follicular lymphoma, cutaneous T-cell lymphoma), pancreatic cancer, bladder cancer, squamous cell carcinoma of the head and neck, genitourinary cancer, advanced cutaneous squamous cell carcinoma, liver metastases, mesothelioma, gastroesophageal cancer, Merkel cell carcinoma, and urothelial carcinoma.

In yet other aspects, the disclosure provides a method of selecting a subject (e.g., a human subject) for treatment, the method comprising determining a level of CTLA-4 in a tumor sample obtained from a subject having cancer and to whom has been administered a primary cell-derived biologic comprising IL-1β, IL-2, IL-6, IL-8, TNF-α, and IFN-γ (e.g., human IL-1β, human IL-2, human IL-6, human IL-8, human TNF-α, and human IFN-γ); and administering to the subject an effective amount of an antagonist of CTLA-4 if the level of CTLA-4 in the tumor sample is higher than a threshold level of CTLA-4. In some embodiments, determining comprises performing an assay to detect the level of CTLA-4. In some embodiments, the assay is selected from the group consisting of in situ hybridization, RT-qPCR, microarray analysis, multiplexed RNA expression analysis, RNA-seq, an immunohistochemistry assay, flow cytometry, a multiplexed protein assay and a Western blot assay.

In some embodiments, the method further comprises administering the primary cell-derived biologic to the subject prior to the determining step. In some embodiments, the antagonist is an antisense oligonucleotide, a short interfering RNA (siRNA), small molecule, a peptide, or an antibody. In some embodiments, the antagonist is an antibody. In some embodiments, the antibody is a human or humanized antibody. In some embodiments, the antibody is selected from the group consisting of ipilimumab and tremelimumab.

In some embodiments, the effective amount of the primary cell-derived biologic administered to the subject includes a ratio of IL-1β International Units (IU) to IL-2 IU of 0.45 to 1.37, a ratio of IFN-γ IU to IL-2 IU of 0.19 to 0.39, a ratio of TNF-α IU to IL-2 IU of 0.53 to 1.26, a ratio of IL-6 IU to IL-2 IU of 1.16 to 6.06, and a ratio of IL-8 IU to IL-2 IU of 0.15 to 0.51. In some embodiments, the effective amount of the primary cell-derived biologic administered to the subject includes at least 1 IU of each of IL-1β, IL-2, IL-6, IL-8, and TNF-α. In some embodiments, the primary cell-derived biologic includes 22-657 International Units (IU) or 220-6,700 pcg of IL-1β, 29-478 IU or 1730-28,100 pcg of IL-2, 10-185 IU or 560-10,900 pcg of IFN-γ, 29-600 IU or 580-12,000 pcg of TNF-α, 34-2,895 IU or 260-22,100 pcg of IL-6 and 5-244 IU or 4,610-243,600 of IL-8. In some embodiments, the primary cell-derived biologic further comprises GM-CSF and G-CSF.

In some embodiments, the cancer is selected from the group consisting of melanoma, lung cancer (such as non-small-cell lung cancer (NSCLC) or SCLC), renal cell carcinoma (RCC), prostate cancer, ovarian cancer, colorectal cancer (CRC), kidney cancer, gastric cancer, breast cancer, diffuse large B cell lymphoma (DLBCL), hematological malignancies (e.g., acute myelogenous leukemia (AML), multiple myeloma (MM), chronic lymphocytic leukemia (CLL), Chronic myelogenous leukemia (CML), Hodgkin's lymphoma, non-Hodgkin's lymphoma, B-cell lymphoma, follicular lymphoma, cutaneous T-cell lymphoma), pancreatic cancer, bladder cancer, squamous cell carcinoma of the head and neck, genitourinary cancer, advanced cutaneous squamous cell carcinoma, liver metastases, mesothelioma, gastroesophageal cancer, Merkel cell carcinoma, and urothelial carcinoma.

In other aspects, method of assessing the likelihood that a subject (e.g., a human subject) will be responsive to an antagonist of CTLA-4, the method comprising administering a primary cell-derived biologic comprising IL-1β, IL-2, IL-6, IL-8, TNF-α, and IFN-γ (e.g., human IL-1β, human IL-2, human IL-6, human IL-8, human TNF-α, and human IFN-γ) to a subject having a cancer that expresses a first level of CTLA-4 that is below a threshold level of CTLA-4; and determining a second level of CTLA-4 in a tumor sample from the subject after administration of the primary cell-derived biologic, wherein a second level of CTLA-4 that is above the threshold level of CTLA-4 is indicative that the subject will be responsive to the antagonist of CTLA-4. In some embodiments, determining comprises performing an assay to detect the second level of CTLA-4. In some embodiments, the assay is selected from the group consisting of in situ hybridization, RT-qPCR, microarray analysis, multiplexed RNA expression analysis, RNA-seq, an immunohistochemistry assay, flow cytometry, a multiplexed protein assay and a Western blot assay. In some embodiments, the cancer is selected from the group consisting of melanoma, lung cancer (such as non-small-cell lung cancer (NSCLC) or SCLC), renal cell carcinoma (RCC), prostate cancer, ovarian cancer, colorectal cancer (CRC), kidney cancer, gastric cancer, breast cancer, diffuse large B cell lymphoma (DLBCL), hematological malignancies (e.g., acute myelogenous leukemia (AML), multiple myeloma (MM), chronic lymphocytic leukemia (CLL), Chronic myelogenous leukemia (CML), Hodgkin's lymphoma, non-Hodgkin's lymphoma, B-cell lymphoma, follicular lymphoma, cutaneous T-cell lymphoma), pancreatic cancer, bladder cancer, squamous cell carcinoma of the head and neck, genitourinary cancer, advanced cutaneous squamous cell carcinoma, liver metastases, mesothelioma, gastroesophageal cancer, Merkel cell carcinoma, and urothelial carcinoma.

In some embodiments of any of the methods provided above, the primary cell-derived biologic may be substituted with a combination of cytokines as described herein (e.g., comprising IL-1β, IL-2, IL-6, IL-8, TNF-α, and IFN-γ), which may be administered together (such as in a cytokine mixture) or separately.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a set of photographs of tumor sections from a patient before treatment with the primary cell-derived biologic treatment regimen described in Example 1 (biopsy) and after treatment with the primary cell-derived biologic treatment regimen (resection). The tumor sections are stained with antibodies to lymphocyte biomarkers including CD68, CD8, CD4, CD8/FOXP3, and CD4/FOXP3. FIG. 1 shows that there is more staining in the resection sample, indicating lymphocyte infiltration of both CD4 and CD8 T cells into the tumor after treatment.

FIG. 2 is a graph showing the difference in mean membrane intensity of PD-L1 expression after treatment of 7 patients treated with the primary cell-derived biologic treatment regimen described in Example 1 (prior to surgery). Each bar represents a single patient. The Y-axis shows the change in mean PD-L1 membrane intensity before and after treatment with the primary cell-derived biologic. FIG. 2 shows that 4 patients had increases in PD-L1 expression after treatment with the primary cell-derived biologic.

FIG. 3 is a graph showing the change in mRNA expression levels of CTLA-4 after treatment of 7 patients treated with a primary cell-derived biologic treatment regimen described in Example 1 (prior to surgery). Each bar represents a single patient. The Y-axis shows the change in CTLA-4 mRNA expression levels before and after treatment with the primary cell-derived biologic. FIG. 3 shows that 5 patients had increases in CTLA-4 expression after treatment with the primary cell-derived biologic.

### DETAILED DESCRIPTION

The present disclosure relates to compositions and methods that utilize a primary cell-derived biologic to induce and/or enhance a therapeutic response to a PD-1/PD-L1 antagonist and/or a CTLA-4 antagonist, to make subjects responsive to treatment with a PD-1/PD-L1 antagonist and/or a CTLA-4 antagonist, or to select subjects for treatments with a PD-1/PD-L1 antagonist and/or a CTLA-4 antagonist.

### Primary Cell-Derived Biologic

In some aspects, the disclosure relates to use of a primary cell-derived biologic, e.g., in a method or composition as described herein. As used herein, the term “primary cell-derived biologic,” is a biologic composition comprising multiple cytokine components, preferably non-recombinant cytokines, that is derived or obtained from primary cells, e.g., human mononuclear cells that have been stimulated with a mitogen and a 4-aminoquinolone antibiotic. An exemplary primary cell-derived biologic is IRX-2 (see, e.g., Egan et al. (2007) J Immunother 30(6):624-633, which describes an exemplary batch of IRX-2 in Table 1 and IRX-2 as described in US Patent Number 8,470,562, each of which are incorporated herein by reference in their entirety). IRX-2 is a primary cell-derived biologic produced by stimulating purified human white blood cells (mononuclear cells) with phytohemagglutinin (PHA) and ciprofloxacin. IRX-2 includes the cytokines: human IL-1β, human IL-2, human IL-6, human IL-8, human TNF-α, and human IFN-γ, which are thought to be the most active components of the biologic, as well as human GM-CSF and human G-CSF. In some embodiments, the primary cell-derived biologic comprises interleukin-lbeta (IL-1β), interleukin-2 (IL-2), interleukin-6 (IL-6), interleukin-8 (IL-8), tumor necrosis factor-alpha (TNF-α), and interferon-gamma (IFN-γ). In some embodiments, the primary cell-derived biologic comprises human IL-1β, human IL-2, human IL-6, human IL-8, human TNF-α, and human IFN-γ. In some embodiments, the primary cell-derived biologic further comprises granulocyte-macrophage colony-stimulating factor (GM-CSF) and granulocyte-colony stimulating factor (G-CSF). In some embodiments, the primary cell-derived biologic further comprises human GM-CSF and human G-CSF.

In some embodiments, the primary cell-derived biologic is delivered to a subject, e.g., in an amount effective to increase PD-L1 and/or CTLA-4 expression. In some embodiments, the amount of primary cell-derived biologic delivered to a subject is defined using International Units (IU) or IU per milliliter (IU/mL) of one or more of the cytokines present in the primary cell-derived biologic. IU and IU/mL for each cytokine are established by the National Institute of Biological Standards and Controls (NIBSC) and assigned a code, which is provided in the below table. Information related to each code can be accessed by referring to the NIBSC website (nibsc.org). IU and IU/mL can be determined by measuring the cytokine units in picograms (pcg) or pcg per milliliter (pcg/mL) using an appropriate R&D Systems test kit provided in the below table, which are converted to IU or IU/mL, respectively, using the conversion factors provided in the below table, which are values derived from the R&D Systems test kit manuals.

| | Conversion Factor: pcg/mL | | |
|---|---|---|---|
| | to IU/mL (multiply pcg/mL | | R&D Systems |
| | by the conversion factor to | NIBSC Standard | Test Kit |
| Cytokine | get IU/mL | Code | Catalog # |
| IL-1β | 0.098 | 86/552 | DLB50 |
| IL-2 | 0.017 | 86/500 | D2050 |
| IFN-γ | 0.017 | 82/587 | DIF50 |
| TNF-α | 0.050 | 88/786 | DTA00C |
| IL-6 | 0.131 | 89/548 | D6050 |
| IL-8 | 0.001 | 89/520 | D8000C |
| G-CSF | 0.120 | 88/502 | DCS50 |
| GM-CSF | 0.008 | 88/646 | DGM00 |

In some embodiments, the amount of the primary cell-derived biologic delivered to a subject contains at least 1 IU (e.g., at least 1 IU, at least 2 IU or at least 3 IU) of each of IL-1β, IL-2, IL-6, IL-8, and TNF-α, and IFN-γ, e.g., human IL-1β, human IL-2, human IL-6, human IL-8, human TNF-α, and human IFN-γ. In some embodiments, the primary cell-derived biologic further comprises GM-CSF and G-CSF, e.g., human GM-CSF and human G-CSF, and the amount of the primary cell-derived biologic delivered to a subject contains at least 0.05 IU (e.g., at least 0.05 IU, at least 0.1 IU or at least 1 IU) of GM-CSF and at least 1 IU (e.g., at least 1 IU, at least 2 IU or at least 3 IU) of G-CSF.

In some embodiments, the amount of the primary cell-derived biologic delivered to a subject contains 22-657 IU (e.g., 30-147 IU) of IL-1β, e.g., human IL-1β; 29-478 IU (e.g., 67-156 IU) of IL-2, e.g., human IL-2; 10-185 IU (e.g., 13-53 IU) of IFN-γ, e.g., human IFN-γ; 29-600 IU (e.g., 36-150 IU) of TNF-α, e.g., human TNF-α; 34-2,895 IU (e.g., 89-524 IU) of IL-6, e.g., human IL-6; and 5-244 IU (e.g., 10-64 IU) of IL-8, human IL-8. In some embodiments, the primary cell-derived biologic further comprises GM-CSF and G-CSF, e.g., human GM-CSF and human G-CSF, and the amount of the primary cell-derived biologic delivered to a subject contains 7-456 IU (e.g., 7-84 IU) of G-CSF and 0.08-28 IU (e.g., 2-6 IU) of GM-CSF.

In some embodiments, the amount of the primary cell-derived biologic delivered to a subject contains a concentration of IL-1β, e.g., human IL-1β, that ranges from 22-657 IU/mL (e.g., from 30-147 IU/mL); a concentration of IL-2, e.g., human IL-2, that ranges from 29-478 IU/mL (e.g., from 67-156 IU/mL); a concentration of IFN-γ, e.g., human IFN-γ, that ranges from 10-185 IU/mL (e.g., from 13-53 IU/mL); a concentration of TNF-α, e.g., human TNF-α, that ranges from 29-600 IU/mL (e.g., from 36-150 IU/mL); a concentration of IL-6, e.g., human IL-6, that ranges from 34-2,895 IU/mL (e.g., 89-524 IU/mL); and a concentration of IL-8, e.g., human IL-8, that ranges from 5-244 IU/mL (e.g., 10-64 IU/mL). In some embodiments, the primary cell-derived biologic further comprises GM-CSF and G-CSF, e.g., human GM-CSF and human G-CSF, and the amount of the primary cell-derived biologic delivered to a subject contains a concentration of G-CSF that ranges from 7-456 IU/mL (e.g., 7-84 IU/mL) and a concentration of GM-CSF that ranges from 0.08-28 IU/mL (e.g., 2-6 IU/mL).

In some embodiments, the amount of the primary cell-derived biologic delivered to a subject contains ratios of each cytokine relative to the amount of IL-2 present in the primary cell-derived biologic. In some embodiments, a lower limit of the ratio for a particular cytokine may be calculated by taking the lowest IU value (e.g., a lowest IU value described herein) for the particular cytokine and dividing it by the lowest IU value (e.g., a lowest IU value described herein) for IL-2. In some embodiments, an upper limit of the ratio for a particular cytokine may be calculated by taking the highest IU value (e.g., a highest IU value described herein) for the particular cytokine and dividing it by the highest IU value (e.g., a highest IU value described herein) for IL-2. In some embodiments, the amount of the primary cell-derived biologic delivered to a subject contains a ratio of IL-1β IU (e.g., human IL-1β IU) to IL-2 IU (e.g., human IL-2 IU) of 0.45 to 1.37 (e.g., 0.45 to 0.94); a ratio of IFN-γ IU (e.g., human IFN-γ IU) to IL-2 IU (e.g., human IL-2 IU) of 0.19 to 0.39 (e.g., 0.19 to 0.34); a ratio of TNF-α IU (e.g., human TNF-α IU) to IL-2 IU (e.g., human IL-2 IU) of 0.53 to 1.26 (e.g., 0.53 to 0.96); a ratio of IL-6 IU (e.g., human IL-6 IU) to IL-2 IU (e.g., human IL-2 IU) of 1.16 to 6.06 (e.g., 1.32 to 3.35); and a ratio of IL-8 IU (e.g., human IL-8 IU) to IL-2 IU (e.g., human IL-2 IU) of 0.15 to 0.51 (e.g., 0.15 to 0.41). In some embodiments, the primary cell-derived biologic further comprises GM-CSF and G-CSF, e.g., human GM-CSF and human G-CSF, and the amount of the primary cell-derived biologic delivered to a subject contains a ratio of G-CSF IU (e.g., human G-CSF IU) to IL-2 IU (e.g., human IL-2 IU) of 0.11 to 0.95 (e.g., 0.11 to 0.54) and a ratio of GM-CSF IU (e.g., human GM-CSF IU) to IL-2 IU (e.g., human IL-2 IU) of 0.002 to 0.06 (e.g., 0.03 to 0.04).

In some embodiments, the amount of primary cell-derived biologic delivered to a subject is defined using pcg or pcg/mL of one or more of the cytokines present in the primary cell-derived biologic. In some embodiments, the amount of the primary cell-derived biologic delivered to a subject contains 220-6,700 pcg (e.g., 310-1,500 pcg) of IL-1β, e.g., human IL-1(3; 1730-28,100 pcg (e.g., 3,960-9,200 pcg) of IL-2, e.g., human IL-2; 560-10,900 pcg (e.g., 750-3,100 pcg) of IFN-γ, e.g., human IFN-γ; 580-12,000 pcg (e.g., 720-3,000 pcg) of TNF-α, e.g., human TNF-α; 260-22,100 pcg (e.g., 680-4,000 pcg) of IL-6, e.g., human IL-6; and 4,610-243,600 pcg (e.g., 10,390-63,800 pcg) of IL-8, e.g., human IL-8. In some embodiments, the primary cell-derived biologic further comprises GM-CSF and G-CSF, e.g., human GM-CSF and human G-CSF, and the amount of the primary cell-derived biologic delivered to a subject contains 60-3,800 pcg (e.g., 60-700 pcg) of G-CSF and 10-3,500 pcg (e.g., 250-800 pcg) of GM-CSF.

In some embodiments, the amount of the primary cell-derived biologic delivered to a subject contains a concentration of IL-1β, e.g., human IL-1β, that ranges from 220-6,700 pcg/mL (e.g., from 310-1,500 pcg/mL); a concentration of IL-2, e.g., human IL-2, that ranges from 1,730-28,100 pcg/mL (e.g., from 3,960-9,200 pcg/mL); a concentration of IFN-γ, e.g., human IFN-γ, that ranges from 560-10,900 pcg/mL (e.g., 750-3,100 pcg/mL); a concentration of TNF-α, e.g., human TNF-α, that ranges from 580-12,000 pcg/mL (e.g., 720-3,000 pcg/mL);a concentration of IL-6, e.g., human IL-6, that ranges from 260-22,100 pcg/mL (e.g., 680-4,000 pcg/mL); and a concentration of IL-8, e.g., human IL-8, that ranges from 4,610-243,600 pcg/mL (e.g., 10,390-63,800 pcg/mL). In some embodiments, the amount of the primary cell-derived biologic delivered to the subject contains a concentration of IL-1β, e.g., human IL-1β, that ranges from 300-1,400 pcg/mL; a concentration of IL-2, e.g., human IL-2, that ranges from 4,000-8,000 pcg/mL; a concentration of IFN-γ e.g., human IFN-γ, that ranges from 1,000-3,800 pcg/mL and a concentration of TNF-α , e.g., human TNF-α, that ranges from 1,000-4,300 pcg/mL. In some embodiments, the primary cell-derived biologic further comprises GM-CSF and G-CSF, e.g., human GM-CSF and human G-CSF, and the amount of the primary cell-derived biologic delivered to a subject contains a concentration of G-CSF that ranges from 60-3,800 pcg/mL (e.g., 60-700 pcg/mL) and a concentration of GM-CSF that ranges from 10-3,500 pcg/mL (e.g., 250-800 pcg/mL).

Exemplary methods of producing a primary cell-derived biologic, such as IRX-2, are disclosed in U.S. Pat. Nos. 8,470,562, 5,632,983 and 5,698,194, all of which are incorporated herein by reference. For example, the primary cell-derived biologic may be prepared by purifying mononuclear cells (MNCs) obtained from human donors, incubating the MNCs overnight, stimulating the MNCs with a mitogen (e.g., continuous or pulsed stimulation with PHA) and 4-aminoquinolone antibiotic (e.g., continuous stimulation with ciprofloxacin) to produce cytokines, removing the mitogen by filtering, clarifying the cytokines by filtering to obtain an initial primary cell-derived biologic supernatant, and separating the initial primary cell-derived biologic supernatant from DNA and adventitious agents using anion exchange chromatography and virus filtration, thereby producing a primary cell-derived biologic, e.g., comprising human IL-1β, human IL-2, human IL-6, human IL-8, human TNF-α, and human IFN-γ.

Other exemplary primary cell-derived biologics and methods of producing primary cell-derived biologics are disclosed, e.g., in U.S. Pat. No. 6,896,879, which is incorporated herein by reference.

### Antagonists of PD-L1 or PD-1

In some aspects, the disclosure relates to antagonists of Programed Cell Death Ligand 1 (PD-L1) or Programmed Cell Death 1 (PD-1) and their use in the compositions and methods described herein.

A PD-1 antagonist, as used herein is an agent that inhibits or prevents PD-1 activity, e.g., by binding to PD-1. A PD-1 antagonist may reduce PD-1 activity in a cell or organism, e.g., by 5%, 10%, 20%, 30%, 40%, 50%, 60%, 70%, 80%, 90%, or 100%, compared to a cell or organism that has not been exposed to the PD-1 antagonist. PD-1 is a cell surface receptor that belongs to the immunoglobulin superfamily and is expressed on T cells, B cells, and macrophages. Human PD-1 is encoded by the gene PDCD1 (Genbank Entrez ID 5133). PD-1 functions as an immune checkpoint and negatively regulates immune responses, e.g. by initiating cell death (apoptosis) and thus inhibiting the activation, expansion, and/or function of CD8⁺ T-cells and other immune cells. PD-L1, a ligand for PD-1, has been found to be highly expressed by several cancers and several PD-1 antagonists are being developed or are approved for treatment of cancer.

PD-1 activity may be interfered with by antibodies that bind selectively to and block the activity of PD-1. The activity of PD-1 can also be inhibited or blocked by molecules other than antibodies that bind PD-1. Such molecules include proteins (such as fusion proteins), small molecules, and peptides, e.g., peptide mimetics of PD-L1 and PD-L2 that bind PD-1 but do not activate PD-1. Agents that bind to and degrade or inhibit the DNA or mRNA encoding PD-1 also can act as PD-1 antagonists. Examples include anti-PD-1 siRNAs and anti-PD-1 antisense oligonucleotides.

Exemplary PD-1 antagonists include those described in U.S. Publications 20130280265, 20130237580, 20130230514, 20130109843, 20130108651,20130017199, 20120251537, and 20110271358, and in European Patent EP2170959B1, the entire disclosures of which are incorporated herein by reference. Other exemplary PD-1 antagonists are described in Curran et al., PNAS, 107, 4275 (2010); Topalian et al., New Engl. J. Med. 366, 2443 (2012); Brahmer et al., New Engl. J. Med. 366, 2455 (2012); Dolan et al., Cancer Control 21, 3 (2014); and Sunshine et al., Curr. Opin. in Pharmacol. 23 (2015).

Exemplary PD-1 antagonists include: nivolumab (e.g., OPDIVO® from Bristol-Myers Squibb), a fully human IgG4 monoclonal antibody that binds PD-1; pidilizumab (e.g., CT-011 from CureTech), a humanized IgG1 monoclonal antibody that binds PD-1; pembrolizumab (e.g., KEYTRUDA® from Merck), a humanized IgG4-kappa monoclonal antibody that binds PD-1; MEDI-0680 (AstraZeneca/MedImmune) a monoclonal antibody that binds PD-1; and REGN2810 (Regeneron/Sanofi) a monoclonal antibody that binds PD-1. Another exemplary PD-1 antagonist is AMP-224 (Glaxo Smith Kline and Amplimmune), a recombinant fusion protein composed of the extracellular domain of the Programmed Cell Death Ligand 2 (PD-L2) and the Fc region of human IgG1, that binds to PD-1.

A PD-L1 antagonist, as used herein is an agent that inhibits or prevents PD-L1 activity, e.g., by binding to PD-L1. A PD-L1 antagonist may reduce PD-L1 activity in a cell or organism, e.g., by 5%, 10%, 20%, 30%, 40%, 50%, 60%, 70%, 80%, 90%, or 100%, compared to a cell or organism that has not been exposed to the PD-L1 antagonist.

PD-L1 is a type 1 transmembrane protein with immunoglobulin V-like and C-like domains. PD-L1 is a ligand for the PD-1 receptor. Human PD-L1 is encoded by the CD274 gene (Genbank Entrez ID 29126). PD-L1 is expressed by both hematopoietic, such as B-cells and T-cells, and non-hematopoietic cells. Binding of PD-L1 to PD-1 results in activation of PD-1, which results in the initiation of cell death (apoptosis) and inhibition of the above-mentioned immune responses, e.g., inhibition of the activation, expansion, and/or function of CD8⁺ T-cells and other immune cells. PD-L1 also binds to CD80 (also known as B7-1).

PD-L1 activity may be blocked by molecules that selectively bind to and block the activity of PD-L1, e.g. by blocking the interaction with and activation of PD-1 and/or B7-1. The activity of PD-L1 can also be inhibited or blocked by molecules other than antibodies that bind PD-L1. Such molecules include proteins (such as fusion proteins), small molecules, and peptides. Agents that bind to and degrade or inhibit the DNA or mRNA encoding PD-L1 also can act as PD-L1 antagonists. Examples include anti-PD-L1 siRNAs and anti-PD-L1 antisense oligonucleotides.

Exemplary PD-L1 antagonists include those described in U.S. Publications 20090055944, 20100203056, 20120039906, 20130045202, 20130309250, and 20160108123, the entire disclosures of which are incorporated herein by reference. Other exemplary PD-L1 antagonists are described in Sunshine et al., Curr. Opin. in Pharmacol. 23 (2015).

PD-L1 antagonists include, for example: atezolizumab (also called MPDL3280A or TECENTRIQ™, Genentech/Roche), an human monoclonal antibody that binds to PD-L1; durvalumab (also called MEDI4736 or IMFINZI™, AstraZeneca/MedImmune), a human immunoglobulin IgG1 kappa monoclonal antibody that binds to PD-L1; BMS-936559 (Bristol-Meyers Squibb), a fully human IgG4 monoclonal antibody that binds to PD-L1; avelumab (also called MSB 0010718C or BAVENCIO®, Merck KGaA/Pfizer), a fully human IgG1 monoclonal antibody that binds to PD-L1; and CA-170 (Aurigene/Curis) a small molecule antagonist of PD-L1.

In some embodiments, the PD-1 or PD-L1 antagonist is an antibody, such a humanized or human antibody. As used herein, the term “antibody” refers to an immunoglobulin molecule that specifically binds to a particular antigen such as PD-L1 or PD-1, and includes polyclonal, monoclonal, genetically engineered and otherwise modified forms of antibodies, including but not limited to chimeric antibodies, humanized antibodies, fully human antibodies, heteroconjugate antibodies (e.g., bispecific antibodies, diabodies, triabodies, and tetrabodies), and antigen binding fragments of antibodies, including e.g., Fab′, F(ab′)2, Fab, Fv, r1gG, and scFv fragments. Moreover, unless otherwise indicated, the term “monoclonal antibody” is meant to include both intact molecules, as well as, antibody fragments (such as, for example, Fab and F(ab′)2 fragments) which are capable of specifically binding to the antigen. An antibody may include an immunoglobulin constant domain from any immunoglobulin, such as IgG1, IgG2, IgG3, or IgG4 subtypes, IgA (including IgA1 and IgA2), IgE, IgD or IgM.

### Antagonists of CTLA-4

In some aspects, the disclosure relates to antagonists of cytotoxic T-lymphocyte-associated protein 4 (CTLA-4) and their use in the compositions and methods described herein.

A CTLA-4 antagonist, as used herein is an agent that inhibits or prevents CTLA-4 activity, e.g., by binding to CTLA-4. A CTLA-4 antagonist may reduce CTLA-4 activity in a cell or organism, e.g., by 5%, 10%, 20%, 30%, 40%, 50%, 60%, 70%, 80%, 90%, or 100%, compared to a cell or organism that has not been exposed to the CTLA-4 antagonist. CTLA-4 (also known as CTLA4 and CD152) is a cell surface receptor that belongs to the immunoglobulin superfamily and is expressed on T cells. Human CTLA-4 is encoded by the gene CTLA4 (Genbank Entrez ID 1493). CTLA-4 functions as an immune checkpoint and negatively regulates immune responses, e.g. by transmitting inhibitory signals to T cells.

CTLA-4 activity may be interfered with by antibodies that bind selectively to and block the activity of CTLA-4. The activity of CTLA-4 can also be inhibited or blocked by molecules other than antibodies that bind CTLA-4. Such molecules include proteins (such as fusion proteins), small molecules, and peptides, e.g., peptide mimetics of CD80 or CD86 that bind CTLA-4 but do not activate CTLA-4. Agents that bind to and degrade or inhibit the DNA or mRNA encoding CTLA-4 also can act as CTLA-4 antagonists. Examples include anti-CTLA-4 siRNAs and anti-CTLA-4 antisense oligonucleotides.

Exemplary CTLA-4 antagonists include those described in PCT Publication Nos. WO2001/014424, WO2012/118750, European Patent No. EP1212422B1, U.S. Pat. Nos. 5,811,097, 5,855,887, 6,051,227, 6,984,720, 7,034,121, 7,824,679, 8,017,114, 8,475,790, 8,318,916, 8,685,394, U.S. Publication Nos. 2002/0039581, 2005/0201994, and 2009/0117037, the entire disclosures of which are incorporated herein by reference. Other exemplary CTLA-4 antagonists are described Hurwitz et al., Proc. Natl. Acad. Sci. USA, 95(17):10067-10071 (1998); Camacho et al., J. Clin. Oncology, 22(145): Abstract No. 2505 (2004) (antibody CP-675206); Mokyr et al., Cancer Res., 58:5301-5304 (1998), and Lipson and Drake, Clin Cancer Res; 17(22) Nov. 15, 2011, all of which are herein incorporated by reference, in their entireties.

Exemplary CTLA-4 antagonists include: ipilimumab (YERVOY®, Bristol-Myers Squibb), which is a recombinant human IgG1 monoclonal antibody against CTLA-4, and tremelimumab/CP-675,206 (Astra7eneca; Medlmmune; Pfizer), which is a human IgG2 monoclonal antibody against CTLA-4.

In some embodiments, the CLTA-4 antagonist is an antibody, such a humanized or human antibody. The CTLA-4 antibody may be any type of antibody, including polyclonal, monoclonal, genetically engineered and otherwise modified forms of antibodies, including but not limited to chimeric antibodies, humanized antibodies, fully human antibodies, heteroconjugate antibodies (e.g., bispecific antibodies, diabodies, triabodies, and tetrabodies), and antigen binding fragments of antibodies, including e.g., Fab′, F(ab′)2, Fab, Fv, r1gG, and scFv fragments.

### Methods of Treatment and Pharmaceutical Compositions

In some aspects, the disclosure relates to methods of treatment, e.g., treatment of cancer or a pre-cancerous lesion. In some embodiments, the method comprises a) administering to a subject having cancer or a pre-cancerous lesion an effective amount of a primary cell-derived biologic as described herein (e.g., comprising IL-1β, IL-2, IL-6, IL-8, TNF-α, and IFN-γ); and b) administering to the subject an effective amount of an antagonist of PD-L1 or PD-1 as described herein. In some embodiments, the method comprises a) administering to a subject having cancer or a pre-cancerous lesion an effective amount of a primary cell-derived biologic as described herein (e.g., comprising IL-1β, IL-2, IL-6, IL-8, TNF-α, and IFN-γ); and b) administering to the subject an effective amount of an antagonist of CTLA-4 as described herein. In some embodiments, the method comprises a) administering to a subject having cancer or a pre-cancerous lesion an effective amount of a primary cell-derived biologic as described herein (e.g., comprising IL-1β, IL-2, IL-6, IL-8, TNF-α, and IFN-γ); and b) administering to the subject an effective amount of an antagonist of CTLA-4 as described herein and an effective amount of an antagonist of PD-L1 or PD-1 as described herein.

In some embodiments, the administration of the primary cell-derived biologic and the PD-L1 or PD-1 antagonist and/or the CTLA-4 antagonist occur separately in time (e.g., where the primary cell-derived biologic and the PD-L1 or PD-1 antagonist and/or the CTLA-4 antagonist are administered as separate compositions and at least one administration of the primary cell-derived biologic occurs at a different time than at least one administration of the PD-L1 or PD-1 antagonist and/or the CTLA-4 antagonist) and/or are administered to different locations in the subject (e.g., by different routes of administration where the primary cell-derived biologic and the PD-L1 or PD-1 antagonist and/or the CTLA-4 antagonist are administered as separate compositions). In some embodiments of the method, when the PD-L1 or PD-1 antagonist and the CTLA-4 antagonist are used in combination, the PD-L1 or PD-1 antagonist and the CTLA-4 antagonist are administered together. In some embodiments, the PD-L1 or PD-1 antagonist and the CTLA-4 antagonist are administered separately in time.

In some embodiments, the primary cell-derived biologic and the PD-L1 or PD-1 antagonist and/or the CTLA-4 antagonist are administered on the same day but to different locations in the subject (e.g., by different routes of administration where the primary cell-derived biologic and the PD-L1 or PD-1 antagonist and/or the CTLA-4 antagonist are administered as separate compositions). In some embodiments, the PD-L1 or PD-1 antagonist is administered intravenously or orally. In some embodiments, the CTLA-4 antagonist is administered intravenously. In some embodiments, the primary cell-derived biologic is administered subcutaneously, perilymphatically (e.g., by subcutaneous injection or catheterization into tissue surrounding a lymph node), by catheter, intranodally, peritumorally, or intratumorally. In some embodiments, the primary cell-derived is administered perilymphatically or intranodally to one or more of the following lymph node beds: axillary, cervical, supraclavicular, infraclavicular, deltoid, inguinal, femoral, mediastinal, subpectoral, internal mammary, and/or retroperitoneal lymph node beds.

In some embodiments, at least one dose of the primary cell-derived biologic is administered before at least one dose of the PD-L1 or PD-1 antagonist. In some embodiments, at least one dose of the primary cell-derived biologic is administered before at least one dose of the CTLA-4 antagonist. In some embodiments, at least one dose of the primary cell-derived biologic is administered before at least one dose of the CTLA-4 antagonist and at least one dose of the PD-L1 or PD-1 antagonist. In some embodiments, at least one dose of the primary cell-derived biologic is administered after at least one dose of the PD-L1 or PD-1 antagonist. In some embodiments, at least one dose of the primary cell-derived biologic is administered after at least one dose of the CTLA-4 antagonist. In some embodiments, at least one dose of the primary cell-derived biologic is administered after at least one dose of the CTLA-4 antagonist and at least one dose of the PD-L1 or PD-1 antagonist. In some embodiments, administration of the primary cell-derived biologic occurs both before and after the PD-L1 or PD-1 antagonist. In some embodiments, administration of the primary cell-derived biologic occurs both before and after the CTLA-4 antagonist. In some embodiments, administration of the primary cell-derived biologic occurs both before and after the CTLA-4 antagonist and the PD-L1 or PD-1 antagonist. In some embodiments, administration of the PD-L1 or PD-1 antagonist occurs both before and after the primary cell-derived biologic. In some embodiments, administration of the CTLA-4 antagonist occurs both before and after the primary cell-derived biologic. In some embodiments, administration of the CTLA-4 antagonist and the PD-L1 or PD-1 antagonist occurs both before and after the primary cell-derived biologic.

In some embodiments, the administration of the primary cell-derived biologic and the PD-L1 or PD-1 antagonist and/or CTLA-4 antagonist occur for multiple cycles. In some embodiments, the primary cell-derived biologic is administered for one or more cycles of up to 10 days (e.g., 4, 5 or 10 days) such as administration once a day for up to 10 days (e.g., once a day for 4, 5 or 10 days), where the days are consecutive or may include one or more (such as 1, 2, 3, 4, or 5) days where the biologic is not delivered, such as during a weekend-day. In some embodiments, the one or more cycles of up to 10 days are part of one or more 21-day cycles involving multiple agents. In some embodiments, for each 21-day cycle, cyclophosphamide is administered on day 1 (e.g., intravenously at 300 mg/m²); indomethacin (e.g., 25 mg orally three times a day), omeprazole (e.g., 20 mg orally) and zinc (e.g., 15 to 30 mg orally) are administered daily for 21 days; and a primary cell-derived biologic as described herein (e.g., comprising IL-1β, IL-2, IL-6, IL-8, TNF-α, and IFN-γ) is administered daily for 4, 5 or 10 days (e.g., either consecutively or as two blocks of consecutive days with one or more days in between) beginning on day 4 of each cycle. An exemplary dosage schedule is shown in the below table. Exemplary 21-day dosage schedule for a primary cell-derived biologic as described herein

| | | Route of | |
|---|---|---|---|
| Agent | Dose | Administration | Treatment Days |
| Cyclophosphamide | 300 mg/m² | IV | 1 |
| Indomethacin | 25 mg TID | Oral | 1-21 |
| | (three times | | |
| | daily) | | |
| Zinc-containing | 1 tab containing | Oral | 1-21 |
| multivitamins | 15 to 30 mg | | |
| | Zinc | | |
| Omeprazole | 20 mg | Oral | 1-21 |
| Primary cell-derived | Dose defined as | Subcutaneous | Any ten days |
| biologic (e.g., IRX-2) | 230 IUs of IL-2 | at or near a | between Day |
| | in biologic, | lymph node | 4 and 15 |
| | administered | regional to | |
| | daily | the cancer | |
| | (Bilateral | | |
| | injections of | | |
| | 115 IUs of IL- | | |
| | 2) | | |

In some embodiments, the PD-L1 or PD-1 antagonist is administered for one or more two to four week cycles where administration of the antagonist occurs once every two to four weeks or daily for each cycle (e.g., once every two weeks, once every three weeks, once every four weeks or daily per cycle). In some embodiments, the CTLA-4 antagonist is administered for one or more three to twelve week cycles where administration of the antagonist occurs once every three to twelve weeks (e.g., once every three weeks, once every four weeks, once every eight weeks or once every twelve weeks).

In some embodiments, a method described herein utilizes a dosage regimen where the primary cell-derived biologic is administered for a cycle of up to 10 days as described above (optionally as part of a 21-day cycle described above) and the PD-L1 or PD-1 antagonist is administered for a two to four week cycle as described above, where the dosage regimen is repeated at least once (e.g., repeated 1, 2, 3 or 4 times per year). In some embodiments of the regimen, the primary cell-derived biologic is administered before the PD-L1 or PD-1 antagonist. In other embodiments of the regimen, the PD-L1 or PD-1 antagonist is administered before the primary cell-derived biologic.

In some embodiments, a method described herein utilizes a dosage regimen where the primary cell-derived biologic is administered for a cycle of up to 10 days as described above (optionally as part of a 21-day cycle described above) and the CTLA-4 antagonist is administered for a three to twelve week cycle as described above, where the dosage regimen is repeated at least once (e.g., repeated 1, 2, 3 or 4 times per year). In some embodiments of the regimen, the primary cell-derived biologic is administered before the CTLA-4 antagonist. In other embodiments of the regimen, the CTLA-4 antagonist is administered before the primary cell-derived biologic.

In some embodiments, a method described herein utilizes a dosage regimen where the primary cell-derived biologic is administered for a cycle of up to 10 days as described above (optionally as part of a 21-day cycle described above), the CTLA-4 antagonist is administered for a three to twelve week cycle as described above, and the PD-L1 or PD-1 antagonist is administered for a two to four week cycle as described above, where the dosage regimen is repeated at least once (e.g., repeated 1, 2, 3 or 4 times per year). In some embodiments of the regimen, the primary cell-derived biologic is administered before the CTLA-4 antagonist and PD-L1 or PD-1 antagonist. In other embodiments of the regimen, the CTLA-4 antagonist and PD-L1 or PD-1 antagonist is administered before the primary cell-derived biologic.

In some embodiments of any of the methods provided herein, the method further comprises administering additional agents. In some embodiments, the additional agent is a chemical inhibitor selected from the group consisting of alkylating agents (e.g., cyclophosphamide), antimetabolites, antibiotics, and immunomodulating agents. In some embodiments, the additional agent is an NSAID selected from the group consisting of indomethacin, ibuprofen, celecoxib, rofecoxib, and combinations thereof. In some embodiments, the additional agent is zinc. In some embodiments, the additional agent is a combination of cyclophosphamide, indomethacin, and zinc.

An “effective amount” of an agent generally refers to an amount sufficient to elicit the desired biological response, e.g., treat the condition. As will be appreciated by those of ordinary skill in this art, the effective amount of an agent described herein may vary depending on such factors as the condition being treated, the mode of administration, and the age, body composition, and health of the subject. The effective amount may encompass an amount that improves overall therapy, reduces or avoids symptoms or causes of the condition, or enhances the therapeutic efficacy of another therapeutic agent.

For treatment of cancer, an effective amount is an amount sufficient to provide a therapeutic benefit in the treatment of the cancer, such as to slow, halt or reverse the growth of cancer cells and/or to kill cancer cells, or to reduce or eliminate one or more symptoms associated with the cancer.

In some embodiments, an effective amount of a primary cell-derived biologic is an amount sufficient to increase a level of PD-L1 expression in a tumor of a subject and/or an amount sufficient to enable and/or enhance the therapeutic efficacy of a PD-1 or PD-L1 antagonist as described herein. In some embodiments, an effective amount of a primary cell-derived biologic is an amount sufficient to increase a level of CTLA-4 expression in a tumor of a subject and/or an amount sufficient to enable and/or enhance the therapeutic efficacy of a CTLA-4 antagonist as described herein. In some embodiments, an effective amount of a primary cell-derived biologic is an amount sufficient to increase a level of PD-L1 and CTLA-4 expression in a tumor of a subject and/or an amount sufficient to enable and/or enhance the therapeutic efficacy of a PD-1 or PD-L1 antagonist and a CTLA-4 antagonist as described herein.

Exemplary effective amounts for antibodies, such as anti-PD-1 and anti-PD-L1 antibodies include 0.01 mg/kg to 20 mg/kg every 1-4 weeks. Other exemplary effective amounts for antibodies, such as anti-CLTA-4 antibodies include 3 mg/kg to 15 mg/kg every 3-12 weeks. In embodiments, such administration is for so long as the disease, e.g., cancer, persists. Examples of dosage regimens and administration routes of exemplary PD-1 and PD-L1 antagonists and CLTA-4 are shown in the below table and are contemplated for use in any method described herein.

| | Dosage regimen (such as amount and | |
|---|---|---|
| PD-1 Antagonist | timing of dosing) | Administration Route |
| Nivolumab | 3 mg/kg once every 2 weeks or 240 mg | Intravenous infusion |
| (OPDIVO ®) | once every 2 weeks | over 60 minutes |
| Pidilizumab (CT-011) | 1.5, 3 mg/kg or 6 mg/kg once every 2 | Intravenous infusion |
| | weeks or every month | over 2 hours |
| Pembrolizumab | 2 mg/kg once every 3 weeks or 200 mg | Intravenous infusion |
| (KEYTRUDA ®) | once every 3 weeks | over 30 minutes |
| MEDI-0680 | Once every two weeks for one year | Intravenous infusion |
| REGN2810 | 3 mg/kg once every two weeks | Intravenous infusion |
| | | over 30 minutes |
| AMP-224 | Up to 10 mg/kg once every two weeks | Intravenous infusion |
| | Dosage regimen (amount and timing | |
| PD-L1 Antagonist | of dosing) | Administration Route |
| Atezolizumab | 1200 mg once every three weeks | Intravenous infusion |
| (TECENTRIQ ™) | | over 60 minutes |
| Durvalumab | 10 mg/kg every two weeks or 1500 mg | Intravenous infusion |
| (IMFINZI ™) | once every 14, 21 or 28 days | over 1 hour |
| BMS-936559 | 0.3, 1, 3 or 10 mg/kg (optionally as an | Intravenous infusion |
| | escalating regimen) once every two | |
| | weeks | |
| Avelumab | 10 mg/kg or 5 mg/kg once every two | Intravenous infusion |
| (BAVENCIO ®) | weeks | over 60 minutes |
| CA-170 | Once a day for 21 days | Orally |
| | Dosage regimen (amount and timing | |
| CTLA-4 Antagonist | of dosing) | Administration Route |
| Ipilimumab | 3 mg/kg once every three weeks (e.g., | Intravenous infusion |
| (YERVOY ®) | up to four doses total) | over 90 minutes |
| Tremelimumab | 15 mg/kg or 75 mg once every four, | Intravenous infusion |
| | eight or 12 weeks (e.g., up to four | |
| | doses total) | |

In some embodiments , the amount of the primary cell-derived biologic delivered to a subject contains at least 1 IU (e.g., at least 1 IU, at least 2 IU or at least 3 IU) of each of IL-1β, IL-2, IL-6, IL-8, and TNF-α, and IFN-γ, e.g., human IL-1β, human IL-2, human IL-6, human IL-8, human TNF-α, and human IFN-γ. In some embodiments, the primary cell-derived biologic further comprises GM-CSF and G-CSF, e.g., human GM-CSF and human G-CSF, and the amount of the primary cell-derived biologic delivered to a subject contains at least 0.05 IU (e.g., at least 0.05 IU, at least 0.1 IU or at least 1 IU) of GM-CSF and at least 1 IU (e.g., at least 1 IU, at least 2 IU or at least 3 IU) of G-CSF. In some embodiments, the amount of the primary cell-derived biologic delivered to a subject contains 22-657 IU (e.g., 30-147 IU) of IL-1β, e.g., human IL-1β; 29-478 IU (e.g., 67-156 IU) of IL-2, e.g., human IL-2; 10-185 IU (e.g., 13-53 IU) of IFN-γ, e.g., human IFN-γ; 29-600 IU (e.g., 36-150 IU) of TNF-α, e.g., human TNF-α; 34-2,895 IU (e.g., 89-524 IU) of IL-6, e.g., human IL-6; and 5-244 IU (e.g., 10-64 IU) of IL-8, e.g., human IL-8. In some embodiments, the primary cell-derived biologic further comprises GM-CSF and G-CSF, e.g., human GM-CSF and human G-CSF, and the amount of the primary cell-derived biologic delivered to a subject contains 7-456 IU (e.g., 7-84 IU) of G-CSF and 0.08-28 IU (e.g., 2-6 IU) of GM-CSF.

In some embodiments, the amount of the primary cell-derived biologic delivered to a subject contains ratios of each cytokine relative to the amount of IL-2 present in the primary cell-derived biologic. In some embodiments, the amount of the primary cell-derived biologic delivered to a subject contains a ratio of IL-1β IU (e.g., human IL-1β IU) to IL-2 IU (e.g., human IL-2 IU) of 0.45 to 1.37 (e.g., 0.45 to 0.94); a ratio of IFN-γ IU (e.g., human IFN-γ IU) to IL-2 IU (e.g., human IL-2 IU) of 0.19 to 0.39 (e.g., 0.19 to 0.34); a ratio of TNF-α IU (e.g., human TNF-α IU) to IL-2 IU (e.g., human IL-2 IU) of 0.53 to 1.26 (e.g., 0.53 to 0.96); a ratio of IL-6 IU (e.g., human IL-6 IU) to IL-2 IU (e.g., human IL-2 IU) of 1.16 to 6.06 (e.g., 1.32 to 3.35); and a ratio of IL-8 IU (e.g., human IL-8 IU) to IL-2 IU (e.g., human IL-2 IU) of 0.15 to 0.51 (e.g., 0.15 to 0.41). In some embodiments, the primary cell-derived biologic further comprises GM-CSF and G-CSF, e.g., human GM-CSF and human G-CSF, and the amount of the primary cell-derived biologic delivered to a subject contains a ratio of G-CSF IU (e.g., human G-CSF IU) to IL-2 IU (e.g., human IL-2 IU) of 0.11 to 0.95 (e.g., 0.11 to 0.54) and a ratio of GM-CSF IU (e.g., human GM-CSF IU) to IL-2 IU (e.g., human IL-2 IU) of 0.002 to 0.06 (e.g., 0.03 to 0.04).

In some embodiments, an effective amount of a primary cell-derived biologic as described herein comprises a concentration of IL-1β, e.g., human IL-1β, that ranges from 22-657 IU/mL (e.g., from 30-147 IU/mL); a concentration of IL-2, e.g., human IL-2, that ranges from 29-478 IU/mL (e.g., from 67-156 IU/mL), a concentration of IFN-γ e.g., human IFN-γ, that ranges from 10-185 IU/mL (e.g., from 13-53 IU/mL); a concentration of TNF-α, e.g., human TNF-α, that ranges from 29-600 IU/mL (e.g., from 36-150 IU/mL); a concentration of IL-6, e.g., human IL-6, that ranges from 34-2,895 IU/mL (e.g., 89-524 IU/mL); and a concentration of IL-8, e.g., human IL-8, that ranges from 5-244 IU/mL (e.g., 10-64 IU/mL). In some embodiments, the primary cell-derived biologic further comprises GM-CSF and G-CSF, e.g., human GM-CSF and human G-CSF, and an effective amount of a primary cell-derived biologic further comprises a concentration of G-CSF that ranges from 7-456 IU/mL (e.g., 7-84 IU/mL) and a concentration of GM-CSF that ranges from 0.08-28 IU/mL (e.g., 2-6 IU/mL). In some embodiments, an effective amount of a primary cell-derived biologic as described herein comprises a concentration of IL-1β, e.g., human IL-1β, that ranges from 300-1,400 pcg/mL; a concentration of IL-2, e.g., human IL-2, that ranges from 4,000-8,000 pcg/mL; a concentration of IFN-γ e.g., human IFN-γ, that ranges from 1,000-3,800 pcg/mL and a concentration of TNF-α , e.g., human TNF-α, that ranges from 1,000-4,300 pcg/mL.

In some embodiments, the amount of the primary cell-derived biologic delivered to a subject contains 220-6,700 pcg (e.g., 310-1,500 pcg) of IL-1β, e.g., human IL-1β; 1,730-28,100 pcg (e.g., 3,960-9,200 pcg) of IL-2, e.g., human IL-2; 560-10,900 pcg (e.g., 750-3,100 pcg) of IFN-γ, e.g., human IFN-γ; 580-12,000 pcg (e.g., 720-3,000 pcg) of TNF-α, e.g., human TNF-α; 260-22,100 pcg (e.g., 680-4,000 pcg) of IL-6, e.g., human IL-6; and 4,610-243,600 pcg (e.g., 10,390-63,800 pcg) of IL-8, e.g., human IL-8. In some embodiments, the primary cell-derived biologic further comprises GM-CSF and G-CSF, e.g., human GM-CSF and human G-CSF, and the amount of the primary cell-derived biologic delivered to a subject contains 60-3,800 pcg (e.g., 60-700 pcg) of G-CSF and 10-3,500 pcg (e.g., 250-800 pcg) of GM-CSF.

In some embodiments, an effective amount of a primary cell-derived biologic as described herein comprises a concentration of IL-1β, e.g., human IL-1β, that ranges from 220-6,700 pcg/mL (e.g., from 310-1,500 pcg/mL); a concentration of IL-2, e.g., human IL-2, that ranges from 1730-28,100 pcg/mL (e.g., from 3,960-9,200 pcg/mL); a concentration of IFN-γ, e.g., human IFN-γ, that ranges from 560-10,900 pcg/mL (e.g., 750-3,100 pcg/mL); a concentration of TNF-α, e.g., human TNF-α, that ranges from 580-12,000 pcg/mL (e.g., 720-3,000 pcg/mL); a concentration of IL-6, e.g., human IL-6, that ranges from 260-22,100 pcg/mL (e.g., 680-4,000 pcg/mL); and a concentration of IL-8, e.g., human IL-8, that ranges from 4,610-243,600 pcg/mL (e.g., 10,390-63,800 pcg/mL). In some embodiments, the primary cell-derived biologic further comprises GM-CSF and G-CSF, e.g., human GM-CSF and human G-CSF, and an effective amount of a primary cell-derived biologic further comprises a concentration of G-CSF that ranges from 60-3,800 pcg/mL (e.g., 60-700 pcg/mL) and a concentration of GM-CSF that ranges from 10-3,500 pcg/mL (e.g., 250-800 pcg/mL).

Any agent described herein (e.g., a primary cell-derived biologic, a PD-1/PD-L1 antagonist, or a CTLA-4 antagonist as described herein) may be formulated as a pharmaceutical composition. The term “pharmaceutical composition” refers to preparations which are in such form as to permit the biological activity of the active ingredients to be effective. In some embodiments, a pharmaceutical composition comprises an agent as described herein (e.g., a primary cell-derived biologic, a PD-1/PD-L1 antagonist, or a CTLA-4 antagonist as described herein) and a pharmaceutically acceptable carrier. As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, albumin, emulsions such as oil/water emulsion, and various types of wetting agents. Preferred diluents for aerosol or parenteral administration are phosphate buffered saline, normal (0.9%) saline, or 5% dextrose. Compositions comprising such carriers are formulated by well-known conventional methods (see, e.g., Remington, The Science and Practice of Pharmacy 20th Ed. Mack Publishing, 2000).

Any agent described herein (e.g., a primary cell-derived biologic, a PD-1/PD-L1 antagonist, or a CTLA-4 antagonist) may be administered by any suitable route as needed for the particular condition being treated. For example, an agent described herein (e.g., a primary cell-derived biologic, a PD-1/PD-L1 antagonist, or a CTLA-4 antagonist as described herein), may be administered parenterally (e.g., intravenous, intraarterial, intraperitoneal, intrathecal, intraventricular, intraurethral, intrasternal, intracranial, intramuscular, intra-ossial, intranodal, intradermal and subcutaneous), peritumoral, intratumoral or orally. In some embodiments, a primary cell-derived biologic as described herein is administered into or near a lymph node, such as by perilymphatic injection, e.g., subcutaneous injection or catheterization into the tissue surrounding a draining lymph node regional to a tumor in the subject. In some embodiments, the primary cell-derived biologic is administered subcutaneously, by catheter, intranodally, peritumorally, or perilymphatically. In some embodiments, the primary cell-derived is administered perilymphatically or intranodally to one or more of the following lymph node beds: axillary, cervical, supraclavicular, infraclavicular, deltoid, inguinal, femoral, mediastinal, subpectoral, internal mammary, and/or retroperitoneal lymph node beds.. In some embodiments, a PD-1/PD-L1 antagonist as described herein is administered intravenously or orally. In some embodiments, a CTLA-4 antagonist as described herein is administered intravenously. It is to be understood, however, that the administration route for an agent described herein (e.g., a primary cell-derived biologic, a PD-1/PD-L1 antagonist, or a CTLA-4 antagonist as described herein) may vary depending on the type of subject being treated, the disease being treated (e.g., the type of cancer), and the severity of the disease.

In some embodiments of any one of the methods described herein, a primary cell-derived biologic and/or the PD-1/PD-L1 antagonist is administered as a neo-adjuvant therapy (e.g., prior to surgery), an adjuvant therapy (e.g., after surgery), or as a treatment for established, recurrent or metastatic disease. In some embodiments of any one of the methods described herein, a primary cell-derived biologic and/or the CTLA-4 antagonist is administered as a neo-adjuvant therapy (e.g., prior to surgery), an adjuvant therapy (e.g., after surgery), or as a treatment for established, recurrent or metastatic disease. In some embodiments of any one of the methods described herein, a primary cell-derived biologic, the CTLA-4 antagonist and the PD-1/PD-L1 antagonist is administered as a neo-adjuvant therapy (e.g., prior to surgery), an adjuvant therapy (e.g., after surgery), or as a treatment for established, recurrent or metastatic disease.

### Method of Selection Subjects or Assessing Likelihood

Other aspects of the disclosure relate to methods of assessment or selection. In some embodiments, a method of selecting a subject for treatment is provided. In some embodiments, the method comprises a) determining a level of PD-L1 and/or CTLA-4 in a tumor sample obtained from a subject having cancer or a pre-cancerous lesion and to whom has been administered a primary cell-derived biologic as described herein (e.g., comprising IL-1β, IL-2, IL-6, IL-8, TNF-α, and IFN-γ); and b) administering to the subject an effective amount of an antagonist of PD-L1 or PD-1 as described herein if the level of PD-L1 in the tumor sample is higher than a threshold level of PD-L1 and/or administering to the subject an effective amount of an antagonist of CTLA-4 as described herein if the level of CTLA-4 in the tumor sample is higher than a threshold level of CTLA-4. In some embodiments, the method further comprises administering a primary cell-derived biologic to the subject prior to the determining step. In some embodiments, the level of PD-L1 and/or CTLA-4 is a protein level. In some embodiments, the level of PD-L1 and/or CTLA-4 is an mRNA level.

In some embodiments, a method of assessing the likelihood that a subject will be responsive to an antagonist of PD-L1 or PD-1 and/or an antagonist of CTLA-4 is provided. In some embodiments, the method comprises a) administering a primary cell-derived biologic described herein (e.g., comprising IL-1β, IL-2, IL-6, IL-8, TNF-α, and IFN-γ) to a subject having a cancer or a pre-cancerous lesion that expresses a first level of PD-L1 and/or CTLA-4 that is below a threshold level of PD-L1 and/or CTLA-4; and b) determining a second level of PD-L1 and/or CTLA-4 in a tumor sample from the subject after administration of a primary cell-derived biologic, wherein a second level of PD-L1 and/or CTLA-4 that is above the threshold level of PD-L1 and/or CTLA-4 is indicative that the subject will be responsive to the antagonist of PD-L1 and/or the antagonist of CTLA-4.

Any appropriate threshold level is contemplated herein. In some embodiments, the threshold level is a level of PD-L1 (e.g., a level of PD-L1 protein on cell membranes, such as tumor cell membranes, immune infiltrate cell membranes, and/or stromal cell membranes) and/or a level of CTLA-4 in a tumor sample from the subject prior to administration of a primary cell-derived biologic as described herein (e.g., comprising IL-1β, IL-2, IL-6, IL-8, TNF-α, and IFN-γ). Accordingly, in some embodiments, the method further comprises determining the threshold level by measuring a level of PD-L1 and/or CTLA-4 in a tumor sample (e.g., a level of PD-L1 expressed by tumor cells in the tumor sample and/or a level of PD-L1 and/or CTLA-4 expressed by infiltrating immune cells in the tumor sample) from the subject prior to administration of a primary cell-derived biologic as described herein (e.g., comprising IL-1β, IL-2, IL-6, IL-8, TNF-α, and IFN-γ). In some embodiments, the threshold level is a pre-defined level of PD-L1 and/or CTLA-4. In some embodiments, the threshold level is a level in a group of subjects, e.g., a group of subjects having cancer or pre-cancerous lesions that are not responsive to a PD-L1 or PD-1 antagonist and/or a CTLA-4 antagonist. In some embodiments, the threshold level is an absence of PD-L1 and/or CTLA-4, e.g., an absence of PD-L1 and/or CTLA-4 in a tumor sample. In some embodiments, the threshold level is the basal standard deviation of variability for the assay used to measure PD-L1 and/or CTLA-4 levels. In some embodiments, the threshold level of PD-L1 is a partial or complete cell membrane staining in 49% of viable tumor cells in the tumor sample. Such a threshold level has been previously described (Garon et al. N Engl J Med. 2015 May 21;372(21):2018-28) and may be determined using, e.g., the commercially available PD-L1 IHC 22C3 pharmDx kit (Dako, Product No. SK00621). In some embodiments, the threshold level is a level of PD-L1 and/or CTLA-4 in a negative control sample, such as a tissue or cell known to be negative for PD-L1, for example the endothelium, fibroblasts, and surface epithelium of a tonsil tissue sample, and/or a tissue or cell known to be negative for CTLA-4, for example non-lymphoid tissue.

In some embodiments of any method described herein, a level of PD-L1 (e.g., a level of PD-L1 mRNA or a level of PD-L1 protein) and/or a level of CTLA-4 (e.g., a level of CTLA-4 mRNA or a level of CTLA-4 protein) is measured using an assay. Any suitable assay is contemplated for use to detect the level of PD-L1 and/or CTLA-4. Exemplary assays are disclosed, e.g., in Current Protocols in Molecular Biology, Wiley Online Library, and other similar databases of protocols. Exemplary assays for detecting PD-L1 and/or CTLA-4 mRNA levels include Northern blot, nuclease protection assay, in situ hybridization, RT-qPCR, microarray analysis, multiplexed RNA expression analysis (e.g., MultiOmyx™ or using barcode-based products available from Nanostring Technologies® or Illumina®) and RNA-sequencing (RNA-seq). Exemplary assays for detecting PD-L1 and/or CTLA-4 protein levels include an immunohistochemistry assay, flow cytometry, a multiplexed protein assay (e.g., MultiOmyx™) or a Western blot assay that utilizes, e.g., an antibody specific for PD-L1 (such as the monoclonal mouse anti-PD-L1, Clone 22C3, available from Dako) or an antibody specific for CTLA-4. In some embodiments, the assay is an immunohistochemistry assay, which may be performed using a kit, e.g., the PD-L1 IHC 22C3 pharmDx kit (Dako, Product No. SK00621). In some embodiments of any of the methods of selection or assessment, the tumor sample is a formalin-fixed sample. For example, a tumor sample is formalin-fixed and paraffin-embedded, the sample is sectioned, the sections are DAB (3,3′-Diaminobenzidine) stained with a monoclonal antibody for PD-L1 (e.g., 22C3 mouse monoclonal antibody) and/or CTLA-4 and counter-stained with Hematoxylin, and the sections are mounted on a slide for assessment. In another example, a tumor sample is formalin-fixed and paraffin-embedded, the sample is sectioned, the sections are fluorescently stained with a monoclonal antibody for PD-L1 and/or CTLA-4, and the sections are analyzed, e.g., using the PerkinElmer OPAL™ system. In another example, a tumor sample is formalin-fixed and paraffin-embedded, the sample is sectioned, the sections are fluorescently stained with a monoclonal antibody for PD-L1 and/or CTLA-4 optionally in combination with staining of other biomarkers, and the sections are analyzed, e.g., using the MultiOmyx™ system available from NeoGenomics Laboratories.

In some embodiments of any of the methods of selection or assessment, a subject is treated with a PD-1 or PD-L1 antagonist as described herein if the subject is selected or the assessment indicates that the subject is likely to respond to a PD-1 or PD-L1 antagonist as described herein. In some embodiments of any of the methods of selection or assessment, a subject is treated with a CTLA-4 antagonist as described herein if the subject is selected or the assessment indicates that the subject is likely to respond to a CTLA-4 antagonist as described herein.

### Subjects

Methods described herein utilize subjects, such as subjects having or suspected of having cancer or a pre-cancerous lesion. In some embodiments, the subject is a mammalian subject such as a human subject having or suspected of having cancer or a pre-cancerous lesions. Other exemplary subjects include non-human primates, pigs, horses, sheep, cows, rabbits, dogs, cats, rats and mice.

In some embodiments, the subject has a tumor that expresses a certain level of PD-L1. In some embodiments, the tumor does not express PD-L1. In some embodiments, the tumor expresses a level of PD-L1 that is below a threshold level as described herein (e.g., partial or complete cell membrane staining in 49% of viable tumor cells in a tumor sample).

In some embodiments, the subject has a tumor that contains infiltrating immune cells that express a certain level of PD-L1 and/or CTLA-4. In some embodiments, the infiltrating immune cells do not express PD-L1 and/or CTLA-4.

In some embodiments, the subject is a subject having cancer, such as a human subject having cancer. In some embodiments, the cancer is selected from the group consisting of melanoma, lung cancer (such as non-small-cell lung cancer (NSCLC) or small-cell lung cancer (SCLC)), renal cell carcinoma (RCC), prostate cancer, ovarian cancer, colorectal cancer (CRC), kidney cancer, gastric cancer, breast cancer, diffuse large B cell lymphoma (DLBCL), hematological malignancies (e.g., acute myelogenous leukemia (AML), multiple myeloma (MM), chronic lymphocytic leukemia (CLL), Chronic myelogenous leukemia (CML), Hodgkin's lymphoma, non-Hodgkin's lymphoma, B-cell lymphoma, follicular lymphoma, cutaneous T-cell lymphoma), pancreatic cancer, bladder cancer, squamous cell carcinoma of the head and neck (H&NSCC also called SCCHN), genitourinary cancer, advanced cutaneous squamous cell carcinoma, liver metastases, mesothelioma, gastroesophageal cancer, Merkel cell carcinoma, and urothelial carcinoma. In some embodiments, the PD-1 or PD-L1 antagonist is selected from the below table and the cancer is a cancer in the table below for the selected PD-1 or PD-L1 antagonist. In some embodiments, the CTLA-4 antagonist is selected from the below table and the cancer is a cancer in the table below for the selected CTLA-4 antagonist.

| | Exemplary Cancers |
|---|---|
| PD-1 Antagonist | |
| Nivolumab | Melanoma, Non-small-cell lung cancer (NSCLC), Renal cell |
| (OPDIVO ®) | carcinoma (RCC), Prostate cancer, Hodgkin lymphoma, Ovarian |
| | cancer, Colorectal cancer (CRC), Genitourinary cancer, Kidney |
| | cancer, Gastric cancer, Triple-negative breast cancer |
| Pidilizumab (CT- | Melanoma, Follicular lymphoma (FL), Diffuse large B cell lymphoma |
| 011) | (DLBCL), Hematological malignancies (AML, NHL, MM, CLL, |
| | Hodgkin lymphoma), Pancreatic cancer |
| Pembrolizumab | Melanoma, NSCLC, Bladder cancer, Hodgkin lymphoma, Breast |
| (KEYTRUDA ®) | cancer, Gastric cancer, Squamous cell carcinoma of the head and neck |
| | (SCCHN), Genitourinary cancer, Urothelial carcinoma |
| MEDI-0680 | Melanoma, clear-cell RCC, B-cell lymphoma |
| REGN2810 | Advanced cutaneous squamous cell carcinoma, Lymphoma |
| AMP-224 | Solid tumor malignancy or cutaneous T-cell lymphoma, Melanoma, |
| | Ovarian cancer, Colorectal cancer |
| PD-L1 Antagonist | |
| Atezolizumab | Melanoma, NSCLC, SCLC, RCC, Bladder cancer, Breast cancer, |
| (TECENTRIQ ™) | Genitourinary cancer, Lymphoma, Multiple Myeloma, Kidney |
| | neoplasms, Head and Neck cancer, Ovarian cancer, Colorectal cancer, |
| | Urothelial carcinoma |
| Durvalumab | NSCLC, SCCHN, Colorectal cancer, Liver metastases, Gastric cancer, |
| (IMFINZI ™) | Breast cancer, Pancreatic cancer, Mesothelioma, Lymphoma, Lung |
| | cancer, Melanoma, Gastroesophageal cancer, Ovarian cancer, |
| | Urothelial carcinoma |
| BMS-936559 | Melanoma, NSCLC, RCC, Ovarian cancer, non-Hodgkin's lymphoma, |
| | Hodgkin lymphoma, Multiple myeloma, Chronic myelogenous |
| | leukemia |
| Avelumab | RCC, NSCLC, Gastric cancer, Hodgkin's lymphoma, Ovarian cancer, |
| (BAVENCIO ®) | Urothelial carcinoma, Merkel cell carcinoma |
| CA-170 | Advanced Solid Tumors, Advanced Lymphomas, melanoma, non- |
| | small cell lung cancer, renal cell carcinoma, Hodgkin lymphoma, triple |
| | negative breast cancer, head and neck cancer, colorectal cancer, gastric |
| | cancer, bladder cancer, and ovarian cancer |
| CTLA-4 Antagonist | |
| Ipilimumab | Metastatic melanoma, lung cancer, prostate cancer, cervical cancer, |
| (YERVOY ®) | colorectal cancer, gastric cancer, pancreatic cancer, ovarian cancer, |
| | urothelial carcinoma |
| Tremelimumab | Lung cancer, B-cell lymphoma, gastric cancer, bladder cancer, head |
| | and neck squamous cell carcinoma, hairy cell leukemia, mesothelioma, |
| | melanoma, breast cancer, renal cell carcinoma, ovarian cancer, |
| | hepatocellular cancer, colorectal cancer |

In some embodiments, the subject is a subject having a pre-cancerous lesion, such as a human subject having a pre-cancerous lesion. In some embodiments, the pre-cancerous lesion is selected from the group consisting of cervical intraepithelial neoplasia (CIN, e.g., CIN Grade III) and vulvar intraepithelial neoplasia (VIN, e.g., VIN Grade III).

In some embodiments, the subject is refractory to a treatment, e.g., treatment with a PD-1 or PD-L1 antagonist as described herein and/or a CTLA-4 antagonist as described herein. A subject may be refractory to a treatment if the condition, e.g., cancer, is resistant to treatment or becomes resistant to treatment over time (e.g., the subject may have been responsive to a PD-1 or PD-L1 antagonist and/or CTLA-4 antagonist as described herein and has become resistant to the antagonist over time). In some embodiments, the subject becomes responsive to treatment after administration of a primary cell-derived biologic as described herein.

### Kits

Other aspects of the disclosure relate to kits, such as kits suitable for performing a method described herein, e.g., treating cancer or a pre-cancerous lesion. In some embodiments, a kit is provided comprising a primary cell-derived biologic as described herein (e.g., comprising IL-1β, IL-2, IL-6, IL-8, TNF-α, and IFN-γ) and an antagonist of PD-L1 or PD-1 as described herein (e.g., nivolumab, pidilizumab, pembrolizumab, MEDI-0680, REGN2810, AMP-224, atezolizumab, durvalumab, BMS-936559, avelumab, or CA-170). In some embodiments, a kit is provided comprising a primary cell-derived biologic as described herein (e.g., comprising IL-1β, IL-2, IL-6, IL-8, TNF-α, and IFN-γ) and an antagonist of CTLA-4 as described herein (e.g., ipilimumab or tremelimumab). In some embodiments, a kit is provided comprising a primary cell-derived biologic as described herein (e.g., comprising IL-1β, IL-2, IL-6, IL-8, TNF-α, and IFN-γ), an antagonist of PD-L1 or PD-1 as described herein (e.g., nivolumab, pidilizumab, pembrolizumab, MEDI-0680, REGN2810, AMP-224, atezolizumab, durvalumab, BMS-936559, avelumab, or CA-170) and an antagonist of CTLA-4 as described herein (e.g., ipilimumab or tremelimumab). In some embodiments, the primary cell-derived biologic is provided in one or more first set of containers and the antagonist of PD-L1 or PD-1 and/or the antagonist of CTLA-4 is provided in one or more second set of containers. In some embodiments, the one or more first set of containers contains a therapeutically effective amount of the primary cell-derived biologic for treating cancer or a pre-cancerous lesion and the one or more second set of containers contains a therapeutically effective amount of the antagonist of PD-L1 or PD-1 and/or the antagonist of CTLA-4 for treating cancer or a pre-cancerous lesion. In some embodiments, the one or more first set of containers contains a concentrated amount of the primary cell-derived biologic that may be diluted on site for administration to a subject or may allow for a smaller volume of the primary cell-derived biologic to be administered to the subject (e.g., a therapeutically effective amount of primary cell-derived biologic described herein may be concentrated by two-fold, three-fold, four-fold, five-fold, ten-fold, 100-fold, 1000-fold or more). In some embodiments, the primary cell-derived biologic, the antagonist of PD-L1 or PD-1 and/or the antagonist of CTLA-4 is frozen or lyophilized. In some embodiments, the kit further comprises instructions for performing a method as described herein, e.g., to treat cancer or a pre-cancerous lesion in a subject. In some embodiments, the kit further comprises one or more delivery devices for administering the primary cell-derived biologic, the antagonist of PD-L1 or PD-1 and/or the antagonist of CTLA-4, such as a syringe or catheter.

### Combinations of Cytokines

In some aspects, the disclosure relates to use of a combination of cytokines (administered separately or together, e.g., in the form of a cytokine mixture), e.g., in a method or composition as described herein. The combination of cytokines may comprise IL-1β, IL-2, IL-6, IL-8, IFN-γ and TNF-α (e.g., human IL-1β, human IL-2, human IL-6, human

IL-8, human IFN-γ and human TNF-α), which can include natural, recombinant or pegylated cytokines or a mixture of natural, recombinant or pegylated cytokines. In some embodiments, the combination of cytokines can further include other natural, recombinant or pegylated cytokines such as GM-CSF and G-CSF (e.g., human GM-CSF and G-CSF). In some embodiments, cytokines may be pegylated in order to increase the half-life of the cytokine in vivo and/or to reduce the immunogenicity or toxicity of the cytokine protein in vivo (see, e.g., United States Patent Application Publication US 2004/0136952).

Exemplary mature human cytokine protein sequences are provided below which may be used to generate cytokines, such as recombinant or pegylated cytokines, to include in the combination of cytokines. Methods for producing combinations of cytokines, such as cytokine mixtures, comprising natural, recombinant, and/or pegylated cytokines are known in the art (see, e.g., U.S. Pat. Nos. 4,738,927, 4,992,367, U.S. Patent Application Publication No. US 2004/0136952 A1 and Mehvar, Modulation of the Pharmacokinetics and Pharmacodynamics of Proteins by Polyethylene Glycol Conjugation, J Pharm Pharmaceut Sci 3(1):125-136 (2000)).

Human IL-1β
(SEQ ID NO: 1)
APVRSLNCTLRDSQQKSLVMSGPYELKALHLQGQDMEQQVVFSMSFVQGE
ESNDKIPVALGLKEKNLYLSCVLKDDKPTLQLESVDPKNYPKKKMEKRFV
FNKIEINNKLEFESAQFPNWYISTSQAENMPVFLGGTKGGQDITDFTMQF
VSS
Human IL-2
(SEQ ID NO: 2)
LSCIALSLALVTNSAPTSSSTKKTQLQLEHLLLDLQMILNGINNYKNPKL
TRMLTFKFYMPKKATELKHLQCLEEELKPLEEVLNLAQSKNFHLRPRDLI
SNINVIVLELKGSETTFMCEYADETATIVEFLNRWITFCQSIIS
Human IL-6
(SEQ ID NO: 3)
VPPGEDSKDVAAPHRQPLTSSERIDKQIRYILDGISALRKETCNKSNMCE
SSKEALAENNLNLPKMAEKDGCFQSGFNEETCLVKIITGLLEFEVYLEYL
QNRFESSEEQARAVQMSTKVLIQFLQKKAKNLDAITTPDPTTNASLLTKL
QAQNQWLQDMTTHLILRSFKEFLQSSLRALRQM
Human IL-8
(SEQ ID NO: 4)
EGAVLPRSAKELRCQCIKTYSKPFHPKFIKELRVIESGPHCANTEIIVKL
SDGRELCLDPKENWVQRVVEKFLKRAENS
Human IFN-γ
(SEQ ID NO: 5)
QDPYVKEAENLKKYFNAGHSDVADNGTLFLGILKNWKEESDRKIMQSQIV
SFYFKLFKNFKDDQSIQKSVETIKEDMNVKFFNSNKKKRDDFEKLTNYSV
TDLNVQRKAIHELIQVMAELSPAAKTGKRKRSQMLFRG
Human TNF-α
(SEQ ID NO: 6)
PVAHVVANPQAEGQLQWLNRRANALLANGVELRDNQLVVPSEGLYLIYSQ
VLFKGQGCPSTHVLLTHTISRIAVSYQTKVNLLSAIKSPCQRETPEGAEA
KPWYEPIYLGGVFQLEKGDRLSAEINRPDYLDFAESGQVYFGII

In some embodiments, the combination of cytokines (which may be delivered separately or together) contains at least 1 IU (e.g., at least 1 IU, at least 2 IU or at least 3 IU) of each of IL-1β, IL-2, IL-6, IL-8, and TNF-α, and IFN-γ, e.g., human IL-1β, human IL-2, human IL-6, human IL-8, human TNF-α, and human IFN-γ. In some embodiments, the combination of cytokines (which may be delivered separately or together) further comprises GM-CSF and G-CSF, e.g., human GM-CSF and human G-CSF, and the combination of cytokines contains at least 0.05 IU (e.g., at least 0.05 IU, at least 0.1 IU or at least 1 IU) of GM-CSF and at least 1 IU (e.g., at least 1 IU, at least 2 IU or at least 3 IU) of G-CSF.

In some embodiments, the combination of cytokines (which may be delivered separately or together) contains ratios of each cytokine relative to the amount of IL-2 delivered. In some embodiments, the combination of cytokines (which may be delivered separately or together) contains a ratio of IL-1β IU (e.g., human IL-1β IU) to IL-2 IU (e.g., human IL-2 IU) of 0.45 to 1.37 (e.g., 0.45 to 0.94); a ratio of IFN-γ IU (e.g., human IFN-γ IU) to IL-2 IU (e.g., human IL-2 IU) of 0.19 to 0.39 (e.g., 0.19 to 0.34); a ratio of TNF-α IU (e.g., human TNF-α IU) to IL-2 IU (e.g., human IL-2 IU) of 0.53 to 1.26 (e.g., 0.53 to 0.96); a ratio of IL-6 IU (e.g., human IL-6 IU) to IL-2 IU (e.g., human IL-2 IU) of 1.16 to 6.06 (e.g., 1.32 to 3.35); and a ratio of IL-8 IU (e.g., human IL-8 IU) to IL-2 IU (e.g., human IL-2 IU) of 0.15 to 0.51 (e.g., 0.15 to 0.41). In some embodiments, the combination of cytokines (which may be delivered separately or together) further comprises GM-CSF and G-CSF, e.g., human GM-CSF and human G-CSF, and the amount of the combination of cytokines delivered to a subject contains a ratio of G-CSF IU (e.g., human G-CSF IU) to IL-2 IU (e.g., human IL-2 IU) of 0.11 to 0.95 (e.g., 0.11 to 0.54) and a ratio of GM-CSF IU (e.g., human GM-CSF IU) to IL-2 IU (e.g., human IL-2 IU) of 0.002 to 0.06 (e.g., 0.03 to 0.04).

In some embodiments, the combination of cytokines (which may be delivered separately or together) contains 22-657 IU (e.g., 30-147 IU) of IL-1β, e.g., human IL-1β; 29-478 IU (e.g., 67-156 IU) of IL-2, e.g., human IL-2; 10-185 IU (e.g., 13-53 IU) of IFN-γ, e.g., human IFN-γ; 29-600 IU (e.g., 36-150 IU) of TNF-α, e.g., human TNF-α; 34-2,895 IU (e.g., 89-524 IU) of IL-6, e.g., human IL-6; and 5-244 IU (e.g., 10-64 IU) of IL-8, e.g., human IL-8. In some embodiments, the combination of cytokines (which may be delivered separately or together) further comprises GM-CSF and G-CSF, e.g., human GM-CSF and human G-CSF, and the combination of cytokines contains 7-456 IU (e.g., 7-84 IU) of G-CSF and 0.08-28 IU (e.g., 2-6 IU) of GM-CSF. In some embodiments, the combination of cytokines (which may be delivered separately or together) contains a concentration of IL-1β, e.g., human IL-1β, that ranges from 22-657 IU/mL (e.g., from 30-147 IU/mL); a concentration of IL-2, e.g., human IL-2, that ranges from 29-478 IU/mL (e.g., from 67-156 IU/mL), a concentration of IFN-γ, e.g., human IFN-γ, that ranges from 10-185 IU/mL (e.g., from 13-53 IU/mL); a concentration of TNF-α, e.g., human TNF-α, that ranges from 29-600 IU/mL (e.g., from 36-150 IU/mL); a concentration of IL-6, e.g., human IL-6, that ranges from 34-2,895 IU/mL (e.g., 89-524 IU/mL); and a concentration of IL-8, e.g., human IL-8, that ranges from 5-244 IU/mL (e.g., 10-64 IU/mL). In some embodiments, the combination of cytokines (which may be delivered separately or together) further comprises GM-CSF and G-CSF, e.g., human GM-CSF and human G-CSF, and the combination of cytokines contains a concentration of G-CSF that ranges from 7-456 IU/mL (e.g., 7-84 IU/mL) and a concentration of GM-CSF that ranges from 0.08-28 IU/mL (e.g., 2-6 IU/mL).

In some embodiments, the combination of cytokines (which may be delivered separately or together) contains 220-6,700 pcg (e.g., 310-1,500 pcg) of IL-1β, e.g., human IL-1β; 1730-28,100 pcg (e.g., 3,960-9,200 pcg) of IL-2, e.g., human IL-2; 560-10,900 pcg (e.g., 750-3,100 pcg) of IFN-γ, e.g., human IFN-γ; 580-12,000 pcg (e.g., 720-3,000 pcg) of TNF-α, e.g., human TNF-α; 260-22,100 pcg (e.g., 680-4,000 pcg) of IL-6, e.g., human IL-6; and 4,610-243,600 pcg (e.g., 10,390-63,800 pcg) of IL-8, e.g., human IL-8. In some embodiments, the combination of cytokines (which may be delivered separately or together) further comprises GM-CSF and G-CSF, e.g., human GM-CSF and human G-CSF, and the combination of cytokines contains 60-3,800 pcg (e.g., 60-700 pcg) of G-CSF and 10-3,500 pcg (e.g., 250-800 pcg) of GM-CSF. In some embodiments, the combination of cytokines (which may be delivered separately or together) contains a concentration of IL-1β, e.g., human IL-1β, that ranges from 300-1,400 pcg/mL; a concentration of IL-2, e.g., human IL-2, that ranges from 4,000-8,000 pcg/mL; a concentration of IFN-γ e.g., human IFN-γ, that ranges from 1,000-3,800 pcg/mL and a concentration of TNF-α , e.g., human TNF-α, that ranges from 1,000-4,300 pcg/mL.

In some embodiments, the combination of cytokines (which may be delivered separately or together) contains a concentration of IL-1β, e.g., human IL-1β, that ranges from 220-6,700 pcg/mL (e.g., from 310-1,500 pcg/mL); a concentration of IL-2, e.g., human IL-2, that ranges from 1730-28,100 pcg/mL (e.g., from 3,960-9,200 pcg/mL); a concentration of IFN-γ, e.g., human IFN-γ, that ranges from 560-10,900 pcg/mL (e.g., 750-3,100 pcg/mL); a concentration of TNF-α, e.g., human TNF-α, that ranges from 580-12,000 pcg/mL (e.g., 720-3,000 pcg/mL); a concentration of IL-6, e.g., human IL-6, that ranges from 260-22,100 pcg/mL (e.g., 680-4,000 pcg/mL); and a concentration of IL-8, e.g., human IL-8, that ranges from 4,610-243,600 pcg/mL (e.g., 10,390-63,800 pcg/mL). In some embodiments, the combination of cytokines (which may be delivered separately or together) further comprises GM-CSF and G-CSF, e.g., human GM-CSF and human G-CSF, and the combination of cytokines contains a concentration of G-CSF that ranges from 60-3,800 pcg/mL (e.g., 60-700 pcg/mL) and a concentration of GM-CSF that ranges from 10-3,500 pcg/mL (e.g., 250-800 pcg/mL).

In some aspects, the disclosure relates to methods of treatment, e.g., treatment of cancer or a pre-cancerous lesion, utilizing a combination of cytokines as described herein. In some embodiments, the method comprises a) administering to a subject having cancer or a pre-cancerous lesion an effective amount of a combination of cytokines as described herein (e.g., comprising IL-1β, IL-2, IL-6, IL-8, TNF-α, and IFN-γ); and b) administering to the subject an effective amount of an antagonist of PD-L1 or PD-1 as described herein. In some embodiments, the method comprises a) administering to a subject having cancer or a pre-cancerous lesion an effective amount of a combination of cytokines as described herein (e.g., comprising IL-1β, IL-2, IL-6, IL-8, TNF-α, and IFN-γ); and b) administering to the subject an effective amount of an antagonist of CTLA-4 as described herein. In some embodiments, the method comprises a) administering to a subject having cancer or a pre-cancerous lesion an effective amount of a combination of cytokines as described herein (e.g., comprising IL-1β, IL-2, IL-6, IL-8, TNF-α, and IFN-γ); and b) administering to the subject an effective amount of an antagonist of CTLA-4 as described herein and an effective amount of an antagonist of PD-L1 or PD-1 as described herein.

In some embodiments, the administration of the combination of cytokines and the PD-L1 or PD-1 antagonist and/or CTLA-4 antagonist occur separately in time and/or are administered to different locations in the subject (e.g., by different routes of administration). In some embodiments of the method, when the PD-L1 or PD-1 antagonist and the CTLA-4 antagonist are used in combination, the PD-L1 or PD-1 antagonist and the CTLA-4 antagonist are administered together. In some embodiments, the PD-L1 or PD-1 antagonist and the CTLA-4 antagonist are administered separately in time.

In some embodiments, the combination of cytokines and the PD-L1 or PD-1 antagonist and/or the CTLA-4 antagonist are administered on the same day but to different locations in the subject (e.g., by different routes of administration). In some embodiments, the PD-L1 or PD-1 antagonist is administered intravenously or orally. In some embodiments, the CTLA-4 antagonist is administered intravenously. In some embodiments, the combination of cytokines is administered subcutaneously, by catheter, intranodally, peritumorally, intratumorally, or perilymphatically (e.g., by subcutaneous injection or catheterization into tissue surrounding a lymph node).

In some embodiments, at least one dose of the combination of cytokines (either separately or together, e.g., as a cytokine mixture) is administered before at least one dose of the PD-L1 or PD-1 antagonist. In some embodiments, at least one dose of the combination of cytokines is administered before at least one dose of the CTLA-4 antagonist. In some embodiments, at least one dose of the combination of cytokines is administered before at least one dose of the CTLA-4 antagonist and at least one dose of the PD-L1 or PD-1 antagonist. In some embodiments, at least one dose of the combination of cytokines (either separately or together, e.g., as a cytokine mixture) is administered after at least one dose of the PD-L1 or PD-1 antagonist. In some embodiments, at least one dose of the combination of cytokines is administered after at least one dose of the CTLA-4 antagonist. In some embodiments, at least one dose of the combination of cytokines is administered after at least one dose of the CTLA-4 antagonist and at least one dose of the PD-L1 or PD-1 antagonist. In some embodiments, administration of the combination of cytokines (either separately or together, e.g., as a cytokine mixture) occurs both before and after the PD-L1 or PD-1 antagonist. In some embodiments, administration of the combination of cytokines occurs both before and after the CTLA-4 antagonist. In some embodiments, administration of the combination of cytokines occurs both before and after the CTLA-4 antagonist and the PD-L1 or PD-1 antagonist. In some embodiments, administration of the PD-L1 or PD-1 antagonist occurs both before and after the combination of cytokines (either separately or together, e.g., as a cytokine mixture). In some embodiments, administration of the CTLA-4 antagonist occurs both before and after the combination of cytokines. In some embodiments, administration of the CTLA-4 antagonist and the PD-L1 or PD-1 antagonist occurs both before and after the combination of cytokines.

In some embodiments, the administration of the combination of cytokines (either separately or together, e.g., as a cytokine mixture) and the PD-L1 or PD-1 antagonist and/or CTLA-4 antagonist occur for multiple cycles. In some embodiments, the combination of cytokines (either separately or together, e.g., as a cytokine mixture) is administered for one or more 10 day cycles such as administration once a day for 10 days, where the 10 days are consecutive or may include one or more (such as 1, 2, 3, 4, or 5) days where the biologic is not delivered, such as during a weekend-day. In some embodiments, the one or more 10-day cycles are part of one or more 21-day cycles involving multiple agents. In some embodiments, for each 21-day cycle, cyclophosphamide is administered on day 1 (e.g., intravenously at 300 mg/m²); indomethacin (e.g., 25 mg orally three times a day), omeprazole (e.g., 20 mg orally) and zinc (e.g., 15 to 30 mg orally) are administered daily for 21 days; and a combination of cytokines (either separately or together, e.g., as a cytokine mixture) as described herein (e.g., comprising IL-1β, IL-2, IL-6, IL-8, TNF-α, and IFN-γ) is administered daily for 10 days (e.g., either consecutively or as two 5-day blocks with one or more days in between) beginning on day 4 of each cycle.

In some embodiments, the PD-L1 or PD-1 antagonist is administered for one or more two to four week cycles where administration of the antagonist occurs once every two to four weeks for each cycle (e.g., once every two weeks, once every three weeks, or once every four weeks per cycle). In some embodiments, the CTLA-4 antagonist is administered for one or more three to twelve week cycles where administration of the antagonist occurs once every three to twelve weeks (e.g., once every three weeks, once every four weeks, once every eight weeks or once every twelve weeks).

In some embodiments, a method described herein utilizes a dosage regimen where the combination of cytokines (either separately or together, e.g., as a cytokine mixture) is administered for a 10 day cycle as described above (optionally as part of a 21-day cycle described above) and the PD-L1 or PD-1 antagonist is administered for a two to four week cycle as described above, where the dosage regimen is repeated at least once (e.g., repeated 1, 2, 3 or 4 times per year). In some embodiments of the regimen, the combination of cytokines (either separately or together, e.g., as a cytokine mixture) is administered before the PD-L1 or PD-1 antagonist. In other embodiments of the regimen, the PD-L1 or PD-1 antagonist is administered before the combination of cytokines (either separately or together, e.g., as a cytokine mixture).

In some embodiments, a method described herein utilizes a dosage regimen where the combination of cytokines (either separately or together, e.g., as a cytokine mixture) is administered for a cycle of up to 10 days as described above (optionally as part of a 21-day cycle described above) and the CTLA-4 antagonist is administered for a three to twelve week cycle as described above, where the dosage regimen is repeated at least once (e.g., repeated 1, 2, 3 or 4 times per year). In some embodiments of the regimen, the combination of cytokines is administered before the CTLA-4 antagonist. In other embodiments of the regimen, the CTLA-4 antagonist is administered before the primary cell-derived biologic.

In some embodiments, a method described herein utilizes a dosage regimen where the combination of cytokines (either separately or together, e.g., as a cytokine mixture) is administered for a cycle of up to 10 days as described above (optionally as part of a 21-day cycle described above), the CTLA-4 antagonist is administered for a three to twelve week cycle as described above, and the PD-L1 or PD-1 antagonist is administered for a two to four week cycle as described above, where the dosage regimen is repeated at least once (e.g., repeated 1, 2, 3 or 4 times per year). In some embodiments of the regimen, the combination of cytokines is administered before the CTLA-4 antagonist and PD-L1 or PD-1 antagonist. In other embodiments of the regimen, the CTLA-4 antagonist and PD-L1 or PD-1 antagonist is administered before the combination of cytokines.

In some embodiments, a kit is provided comprising a combination of cytokines (either separately or together, e.g., as a cytokine mixture) as described herein (e.g., comprising IL-1β, IL-2, IL-6, IL-8, TNF-α, and IFN-γ) and an antagonist of PD-L1 or PD-1 as described herein (e.g., nivolumab, pidilizumab, pembrolizumab, MEDI-0680, REGN2810, AMP-224, atezolizumab, durvalumab, BMS-936559, avelumab, or CA-170). In some embodiments, a kit is provided comprising a combination of cytokines (either separately or together, e.g., as a cytokine mixture) as described herein (e.g., comprising IL-1β, IL-2, IL-6, IL-8, TNF-α, and IFN-γ) and an antagonist of CTLA-4 as described herein (e.g., ipilimumab or tremelimumab). In some embodiments, a kit is provided comprising a combination of cytokines (either separately or together, e.g., as a cytokine mixture), an antagonist of PD-L1 or PD-1 as described herein (e.g., nivolumab, pidilizumab, pembrolizumab, MEDI-0680, REGN2810, AMP-224, atezolizumab, durvalumab, BMS-936559, avelumab, or CA-170) and an antagonist of CTLA-4 as described herein (e.g., ipilimumab or tremelimumab). In some embodiments, the combination of cytokines (either separately or together, e.g., as a cytokine mixture) is provided in one or more first set of containers and the antagonist of PD-L1 or PD-1 and/or antagonist of CTLA-4 is provided in one or more second set of containers. In some embodiments, the one or more first set of containers contains a therapeutically effective amount of the combination of cytokines (either separately or together, e.g., as a cytokine mixture) for treating cancer or a pre-cancerous lesion and the one or more second set of containers contains a therapeutically effective amount of the antagonist of PD-L1 or PD-1 and/or antagonist of CTLA-4 for treating cancer or a pre-cancerous lesion. In some embodiments, the combination of cytokines (either separately or together, e.g., as a cytokine mixture), the antagonist of PD-L1 or PD-1 and/or antagonist of CTLA-4 is frozen or lyophilized. In some embodiments, the kit further comprises instructions for performing a method as described herein, e.g., to treat cancer or a pre-cancerous lesion in a subject. In some embodiments, the kit further comprises one or more delivery devices for administering the combination of cytokines (either separately or together, e.g., as a cytokine mixture), the antagonist of PD-L1 or PD-1 and/or antagonist of CTLA-4, such as a syringe or catheter.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

### A Primary Cell-Derived Biologic Increases Lymphocyte Infiltration and PD-L1 Expression

The treatment of cancer has recently advanced with the emergence of cancer immunotherapy. Checkpoint inhibitors (CIs) are now a fundamental new modality to treat cancer along with the more established modalities of surgery, radiotherapy and chemotherapy and offer new therapeutic hope for many patients. Pembrolizumab and Nivolumab were approved for first line metastatic melanoma, metastatic melanoma that has failed therapy with a B-raf inhibitor or Ipilimumab, and treatment of non-small cell lung cancer that has failed a platinin-based therapy. Recently, Pembrolizumab was approved for second-line treatment of renal cancer, and Atezolizumab (TECENTRIQ™) was the first PD-L1 inhibitor approved for bladder cancer. Durvalumab also received break-through designation for inoperable or recurrent metastatic bladder cancer.

IRX-2 is a primary cell-derived biologic with multiple cytokine components generated from donor peripheral blood mononuclear cells stimulated with a strong immunogen (PHA). The IRX-2 biologic contains multiple cytokines, comprising IL-1β, IL-2, IL-6, IL-8, TNF-α, and IFN-γ, that work together synergistically to generate a strong immune response. IRX-2 has multiple effects on the different cells of the immune system including activating and enhancing antigen presentation by antigen presenting cells, increasing the proliferation and cytolytic capability of T cells and protecting them from apoptosis, and increasing the number and activity of NK cells (Egan et al. (2007) J Immunother 30(6):624-633; Czystowska et al. (2009) Cell Death Differ 16(5):708-718; Czystowska et al. (2011) Cancer immunology, immunotherapy : CII 60(4):495-506; Schilling et al. (2012) Cancer immunology, immunotherapy : CII 61(9):1395-1405; Schilling et al. (2013) PLoS One 8(2):e47234; and Schilling et al. (2012) J Mol Med (Berl) 90(2):139-147)). In the clinic, the IRX-2 regimen (which utilizes IRX-2, cyclophosphamide, indomethacin and zinc) has been shown to be safe with a favorable toxicity profile (Freeman et al. (2011) Am J Clin Oncol 34(2):173-178). Important clinical proof of concept was obtained through detailed analysis of the pre- and post-treatment tumor specimens from a multi-center phase 2a trial where increases in lymphocyte infiltration after the IRX-2 regimen were seen in 21 of 25 evaluable patients (Berinstein et al. (2012) Cancer immunology, immunotherapy. Increased lymphocyte infiltration in patients with head and neck cancer treated with the IRX-2 immunotherapy regimen. 61(6):771-782). 7 subjects treated in the above phase 2a trial were analyzed retrospectively for expression of further biomarkers to characterize the infiltrate and elucidate the suppressive mechanisms within the tumor microenvironment that can inform immune interventions that may augment tumor-specific immune response. The IRX-2 regimen (as shown in the table below) given to the subjects was a 21-day regimen including IRX-2 daily for 10 days between Days 4 and 15, cyclophosphamide on Day 1, and indomethacin, zinc, and a proton pump inhibitor on Days 1-21.

| | | Route of | |
|---|---|---|---|
| Agent | Dose | Administration | Treatment Days |
| IRX-2 | 230 units | Subcutaneous | Any ten days |
| | daily | | between Days 4 |
| | Bilateral | | and 15 (For example, |
| | injections of | | Days 4-8 and 11-15) |
| | 115 units | | |
| Cyclophosphamide | 300 mg/m² | IV | 1 |
| Indomethacin | 25 mg TID | Oral | 1-21 |
| Zinc-containing | 1 tab | Oral | 1-21 |
| multivitamins | | | |
| Proton pump | Therapeutic | Oral | 1-21 |
| inhibitor | dose | | |

The IRX-2 dosage was defined by the presence of 115 IL-2 International Units (IU) in the dose delivered to the subject in each injection.

Formalin fixed paraffin embedded (FFPE) samples of patients' biopsy (before treatment with IRX-2) were compared to primary tumor resection samples (after treatment with IRX-2) and stained for markers including CD4, CD8, PD-L1, CD68, FoxP3 and cytokeratin using the PerkinElmer OPAL™ system. For PD-L1 expression rabbit monoclonal antibody clone SP142 from Spring Bio was used for staining. PD-L1 expression was measured on cell membranes and reported as mean normalized fluorescence. Gating settings for identification of four levels (bins) of membrane fluorescence for PD-L1 cell populations were established within tumor tissue. Using these gating settings, cells in the tumor were assigned to one of the four PD-L1 expression bins (0-3+). Results were presented as histograms and used to compute an H-score for PD-L1 in tumor. Mean membrane PD-L1 intensity was also reported for CD68+ cells in tumor and non-tumor tissue segments.

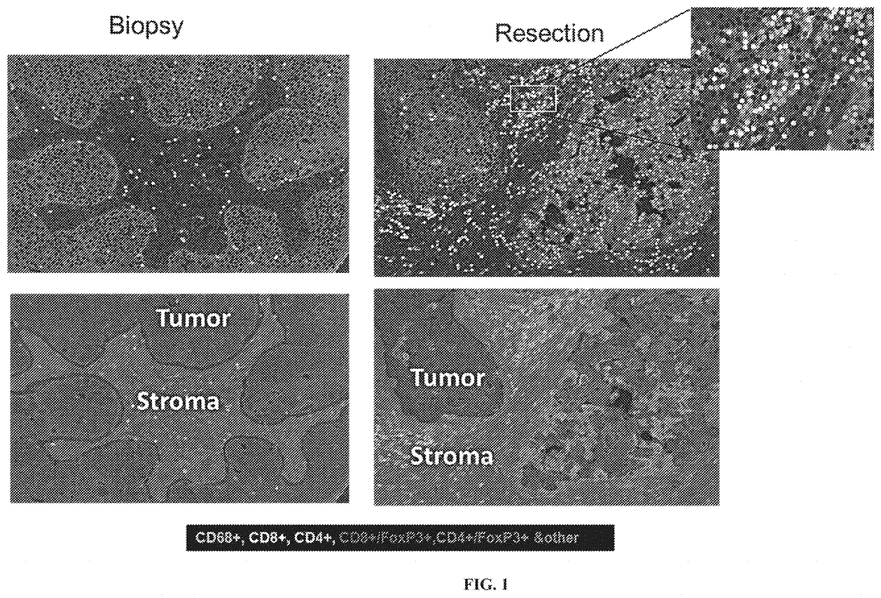

Analysis of the 7 matched patients by multiplex immunohistochemistry (IHC) confirmed previous results, using H&E staining, that IRX-2 treatment resulted in increased lymphocyte infiltration (LI) as well as increases in macrophage infiltration (FIG. 1 shows one exemplary patient).

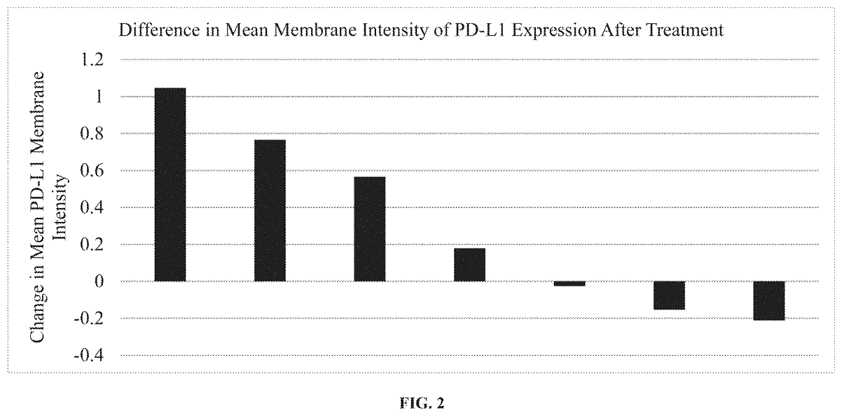

This system was then used to evaluate expression and changes in expression of the PD-L1 check-point pathway. In 4 of 7 of the patients, there were meaningful increases in PD-L1 after IRX-2 treatment (FIG. 2). Thus IRX-2 increases lymphocyte infiltration and PD-L1 expression, suggesting that the IRX-2 regimen may be initiating a tumor-specific immune response.

Although results with checkpoint inhibitors in a number of different tumor types are promising and have provided proof of concept for immunotherapeutic approaches, a plateau has been reached whereby only around 20-30% of treated patients benefit from checkpoint inhibition. The benefit has been further improved by combining different checkpoint inhibitors but unfortunately this approach is associated with an increased toxicity profile (Postow et al. (2015) The New England Journal of Medicine 372(21):2006-2017). Biomarkers of response such as LI, PD-L1 expression, mutational load and status of mismatch repair enzymes have been used to select for patients more likely to respond (Rizvi et al. (2015) Cancer immunology. Science 348(6230):124-128; Garon et al. (2015) The New England Journal of Medicine 372(21):2018-2028; and Le et al. (2015) The New England Journal of Medicine 372(26):2509-2520). However, the fundamental challenge is to develop strategies to increase PD-L1 expression in the tumor, as PD-L1 expression in the tumor appears to be the single most important factor for the clinical outcome of patients treated with such inhibitors (see, e.g., Taube et al., Clin Cancer Res; 20(19): 5064-74 (2014); Sunshine and Taube, Current Opinion in Pharmacology, 23:32-38 (2015); and Carbognin et al. PLoS ONE 10(6): e0130142. (2015)). The data herein show that the expression of PD-L1 was increased in several patients after administration of IRX-2. This suggests that application of a PD-1/PD-L1 inhibitor after IRX-2 treatment may increase the therapeutic activity of the PD-1/PD-L1 inhibitor and/or increase the patient population that is capable of responding to PD-1/PD-L1 inhibition.

### Example 2

### A Primary Cell-Derived Biologic Increases CTLA-4 Expression

The 7 subjects treated in the phase 2a trial described in Example 1 above were also analyzed retrospectively for expression of CTLA-4.

Formalin fixed paraffin embedded (FFPE) samples of patients' biopsy (before treatment with IRX-2) were compared to primary tumor resection samples (after treatment with IRX-2). One FFPE tissue slide was stained with hematoxylin and eosin (HE) and reviewed by a pathologist to delineate the tumor area. The mean tumor cell content was 60% (minimum 30%). MiRNA or total RNA was isolated from the tumor area of 5-μm slices by High Pure microRNA FFPE Isolation Kit (Roche, Basel, Switzerland) or High Pure FFPE RNA Micro Kit (Roche) according to the manufacturer's protocols. Briefly, tissue sections were first deparaffinized with xylene and washed with ethanol. The tissues were then lysed and treated with proteinase K for 3 hours at 55° C. Thereafter, lysates were applied onto spin columns and after a washing step miRNA was eluted in 50 μl elution buffer. Total RNA was eluted twice in 40 μl elution buffer. Afterwards, the RNA was purified and concentrated using the Clean&Concentrator-5™ Kit (Zymo Research, Irvine, Calif., USA) according to the manufacturer's protocol. RNA yield was measured by NanoDrop™ 2000 (Implen GmbH, Munich, Germany) or Qubit® RNA BR Assay Kit (Thermo Fisher Scientific, Waltham, Mass., USA) on the Qubit® 3.0 Fluorometer (Thermo Fisher Scientific). RNA quality was determined on a Lab-on-a-Chip 2100 Bioanalyzer (Agilent Technologies, Santa Clara, Calif., USA). As RNA from FFPE material may possess low quality (RIN values <2), samples were not excluded solely based on RIN (RNA Integrity Number) values. CTLA-4 mRNA levels from the total RNA were measured using the nCounter® PanCancer Immune Profiling Panel (Nanostring Technologies, Seattle, Wash., USA) according to the manufacturer's specifications.

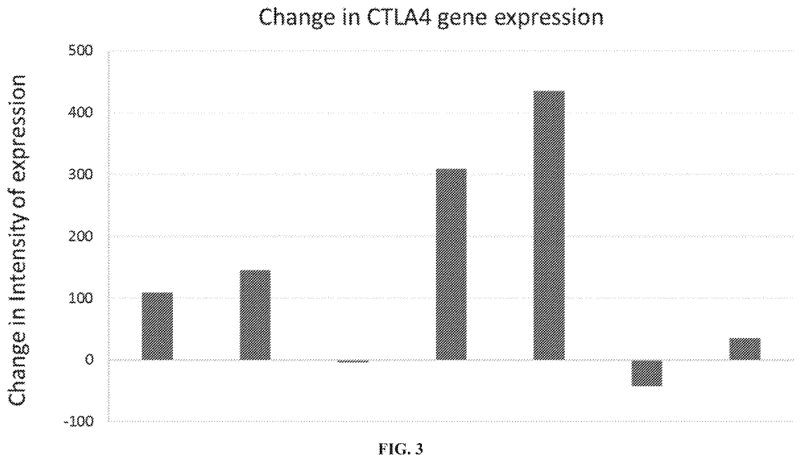

This system was then used to evaluate expression and changes in expression of the CTLA-4 check-point pathway. In 5 of 7 of the patients, there were meaningful increases in CTLA-4 gene expression after IRX-2 treatment (FIG. 3). Thus IRX-2 increases CTLA-4 expression, further suggesting that the IRX-2 regimen may be initiating a functional tumor-specific immune response and physiologic induction of the CTLA4 immune regulatory pathway.

It has been shown previously that increased pretreatment levels of CTLA-4 are predictive of a positive clinical response to CTLA4 blockade (see, e.g., Jamieson et al. Gene-expression profiling to predict responsiveness to immunotherapy. Cancer Gene Therapy (2017) 24:134-140 and Van Allen et al. Genomic correlates of response to CTLA-4 blockade in metastatic melanoma. Science (2015) 350(6257):207-211). The data herein show that the expression of CTLA-4 was increased in several patients after administration of IRX-2. This suggests that application of a CTLA-4 inhibitor after IRX-2 treatment may increase the therapeutic activity of the CTLA-4 inhibitor and/or increase the patient population that is capable of responding to CTLA-4 inhibition.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of treating cancer in a subject, the method comprising:
a) administering to a subject having cancer an effective amount of a primary cell-derived biologic comprising IL-1β, IL-2, IL-6, IL-8, TNF-α, and IFN-γ; and
b) administering to the subject an effective amount of an antagonist of programmed cell death-ligand 1 (PD-L1) or programmed cell death 1 (PD-1), wherein the administration of the primary cell-derived biologic and the administration of the antagonist occur at different locations in the subject and/or at different times.

2. The method of claim 1, wherein at least one administration of the primary cell-derived biologic occurs prior to at least one administration of the antagonist.

3. The method of claim 2, wherein at least one administration of the primary cell-derived biologic occurs prior to at least one administration of the antagonist and at least one further administration of the primary cell-derived biologic occurs after the at least one administration of the antagonist.

4. The method of claim 1, wherein at least one administration of the antagonist occurs prior to at least one administration of the primary cell-derived biologic.

5. The method of claim 4, wherein at least one administration of the antagonist occurs prior to at least one administration of the primary cell-derived biologic and at least one further administration of the antagonist occurs after the at least one administration of the primary cell-derived biologic.

6. The method of any one of claims 1 to 5, wherein the primary cell-derived biologic is administered subcutaneously or perilymphatically and the antagonist is administered intravenously or orally.

7. The method of any one of claims 1 to 6, wherein the primary cell-derived biologic is administered once a day up to 10 days and the antagonist of PD-L1 or PD-1 is administered once every two to four weeks.

8. The method of any one of claims 1 to 7, wherein the antagonist is an antisense oligonucleotide, a short interfering RNA (siRNA), small molecule, a peptide, or an antibody.

9. The method of claim 8, wherein the antagonist is an antibody.

10. The method of claim 9, wherein the antibody is a human or humanized antibody.

11. The method of claim 9 or 10, wherein the antibody is specific for PD-L1.

12. The method of claim 11, wherein the antibody is selected from the group consisting of atezolizumab, durvalumab, BMS-936559, and avelumab.

13. The method of claim 8, wherein the antagonist is CA-170.

14. The method of claim 9 or 10, wherein the antibody is specific for PD-1.

15. The method of claim 14, wherein the antibody is selected from the group consisting of nivolumab, pidilizumab, pembrolizumab, MEDI-0680, and REGN2810.

16. The method of claim 8, wherein the antagonist is AMP-224.

17. The method of any one of claims 1 to 16, wherein the subject is refractory to treatment with the antagonist prior to administration of the primary cell-derived biologic.

18. The method of any one of claims 1 to 17, wherein a level of PD-L1 in a tumor of the subject increases after administration of the primary cell-derived biologic.

19. A method of treating cancer in a subject, the method comprising:
a) administering to a subject having cancer an effective amount of a primary cell-derived biologic comprising IL-1β, IL-2, IL-6, IL-8, TNF-α, and IFN-γ; and
b) administering to the subject an effective amount of an antagonist of programmed cell death-ligand 1 (PD-L1) or programmed cell death 1 (PD-1), wherein the antagonist is selected from the group consisting of nivolumab, pidilizumab, pembrolizumab, MEDI-0680, REGN2810, AMP-224, atezolizumab, durvalumab, BMS-936559, avelumab, and CA-170.

20. The method of any one of claims 1 to 19, wherein the effective amount of the primary cell-derived biologic administered to the subject includes a ratio of IL-1β International Units (IU) to IL-2 IU of 0.45 to 1.37, a ratio of IFN-γ IU to IL-2 IU of 0.19 to 0.39, a ratio of TNF-α IU to IL-2 IU of 0.53 to 1.26, a ratio of IL-6 IU to IL-2 IU of 1.16 to 6.06, and a ratio of IL-8 IU to IL-2 IU of 0.15 to 0.51.

21. The method of any one of claims 1 to 20, wherein the effective amount of the primary cell-derived biologic administered to the subject includes at least 1 IU of each of IL-1β, IL-2, IL-6, IL-8, and TNF-α.

22. The method of any one of claims 1 to 21, wherein the effective amount of the primary cell-derived biologic administered to the subject includes 22-657 International Units (IU) or 220-6,700 pcg of IL-1(3, 29-478 IU or 1730-28,100 pcg of IL-2, 10-185 IU or 560-10,900 pcg of IFN-γ, 29-600 IU or 580-12,000 pcg of TNF-α, 34-2,895 IU or 260-22,100 pcg of IL-6 and 5-244 IU or 4,610-243,600 of IL-8.

23. The method of any one of claims 1 to 22, wherein the primary cell-derived biologic further comprises GM-CSF and G-CSF.

24. The method of any one of claims 1 to 23, further comprising administering to the subject a chemical inhibitor selected from the group consisting of alkylating agents, antimetabolites, antibiotics, and immunomodulating agents.

25. The method of claim 24, wherein the alkylating agent is cyclophosphamide.

26. The method of any one of claims 1 to 25, further comprising administering to the subject an NSAID selected from the group consisting of indomethacin, ibuprofen, celecoxib, rofecoxib, and combinations thereof.

27. The method of claim 26, wherein the NSAID is indomethacin.

28. The method of any one of claims 1 to 27, further comprising administering zinc to the subject.

29. The method of any one of claims 1 to 28 further comprising administering an effective amount of a cytotoxic T-lymphocyte-associated protein 4 (CTLA-4) antagonist.

30. The method of claim 29, wherein the CTLA-4 antagonist is an antisense oligonucleotide, a short interfering RNA (siRNA), small molecule, a peptide, or an antibody.

31. The method of claim 30, wherein the CTLA-4 antagonist is an antibody.

32. The method of claim 31, wherein the antibody is a human or humanized antibody.

33. The method of claim 32, wherein the antibody is selected from the group consisting of ipilimumab and tremelimumab.

34. A method of selecting a subject for treatment, the method comprising:
a) determining a level of PD-L1 in a tumor sample obtained from a subject having cancer and to whom has been administered a primary cell-derived biologic comprising IL-1β, IL-2, IL-6, IL-8, TNF-α, and IFN-γ; and
b) administering to the subject an effective amount of an antagonist of PD-L1 or PD-1 if the level of PD-L1 in the tumor sample is higher than a threshold level of PD-L1.

35. The method of claim 34, wherein determining comprises performing an assay to detect the level of PD-L1.

36. The method of claim 35, wherein the assay is selected from the group consisting of in situ hybridization, RT-qPCR, microarray analysis, multiplexed RNA expression analysis, RNA-seq, an immunohistochemistry assay, flow cytometry, a multiplexed protein assay and a Western blot assay.

37. The method of any one of claims 34 to 36, wherein the level of PD-L1 in the tumor sample is a level of PD-L1 in cell membranes in the tumor sample.

38. The method of claim 37, wherein determining comprises performing an immunohistochemistry assay and the threshold level of PD-L1 is partial or complete cell membrane staining in 49% of viable tumor cells in the tumor sample.

39. The method of any one of claims 34 to 38, further comprising administering the primary cell-derived biologic to the subject prior to the determining step.

40. The method of any one of claims 34 to 39, wherein the antagonist is an antisense oligonucleotide, a short interfering RNA (siRNA), small molecule, a peptide, or an antibody.

41. The method of claim 40, wherein the antagonist is an antibody.

42. The method of claim 41, wherein the antibody is a human or humanized antibody.

43. The method of claim 41 or 42, wherein the antibody is specific for PD-L1.

44. The method of claim 43, wherein the antibody is selected from the group consisting of atezolizumab, durvalumab, BMS-936559, and avelumab.

45. The method of claim 40, wherein the antagonist is CA-170.

46. The method of claim 41 or 42, wherein the antibody is specific for PD-1.

47. The method of claim 46, wherein the antibody is selected from the group consisting of nivolumab, pidilizumab, pembrolizumab, MEDI-0680, and REGN2810.

48. The method of claim 40, wherein the antagonist is AMP-224.

49. The method of any one of claims 34 to 48, wherein the effective amount of the primary cell-derived biologic administered to the subject includes a ratio of IL-1β International Units (IU) to IL-2 IU of 0.45 to 1.37, a ratio of IFN-γ IU to IL-2 IU of 0.19 to 0.39, a ratio of TNF-α IU to IL-2 IU of 0.53 to 1.26, a ratio of IL-6 IU to IL-2 IU of 1.16 to 6.06, and a ratio of IL-8 IU to IL-2 IU of 0.15 to 0.51.

50. The method of any one of claims 34 to 49, wherein the effective amount of the primary cell-derived biologic administered to the subject includes at least 1 IU of each of IL-1β, IL-2, IL-6, IL-8, and TNF-α.

51. The method of any one of claims 34 to 50, wherein the primary cell-derived biologic includes 22-657 International Units (IU) or 220-6,700 pcg of IL-1β, 29-478 IU or 1730-28,100 pcg of IL-2, 10-185 IU or 560-10,900 pcg of IFN-γ, 29-600 IU or 580-12,000 pcg of TNF-α, 34-2,895 IU or 260-22,100 pcg of IL-6 and 5-244 IU or 4,610-243,600 of IL-8.

52. The method of any one of claims 34 to 51, wherein the primary cell-derived biologic further comprises GM-CSF and G-CSF.

53. A method of assessing the likelihood that a subject will be responsive to an antagonist of PD-L1 or PD-1, the method comprising:
a) administering a primary cell-derived biologic comprising IL-1β, IL-2, IL-6, IL-8, TNF-α, and IFN-γ to a subject having a cancer that expresses a first level of PD-L1 that is below a threshold level of PD-L1; and
b) determining a second level of PD-L1 in a tumor sample from the subject after administration of the primary cell-derived biologic, wherein a second level of PD-L1 that is above the threshold level of PD-L1 is indicative that the subject will be responsive to the antagonist of PD-L1 or PD-1.

54. The method of claim 53, wherein determining comprises performing an assay to detect the second level of PD-L1.

55. The method of claim 54, wherein the assay is selected from the group consisting of in situ hybridization, RT-qPCR, microarray analysis, multiplexed RNA expression analysis, RNA-seq, an immunohistochemistry assay, flow cytometry, a multiplexed protein assay and a Western blot assay.

56. The method of any one of claims 53 to 55, wherein the second level of PD-L1 is a level of PD-L1 in cell membranes in the tumor sample.

57. The method of claim 56, wherein determining comprises performing an immunohistochemistry assay and the threshold level of PD-L1 is partial or complete cell membrane staining of at least 49% of viable tumor cells in the tumor sample.

58. The method of any one of claims 1 to 57, wherein the cancer is selected from the group consisting of melanoma, lung cancer (such as non-small-cell lung cancer (NSCLC)), renal cell carcinoma (RCC), prostate cancer, ovarian cancer, colorectal cancer (CRC), kidney cancer, gastric cancer, breast cancer, diffuse large B cell lymphoma (DLBCL), hematological malignancies (e.g., acute myelogenous leukemia (AML), multiple myeloma (MM), chronic lymphocytic leukemia (CLL), Chronic myelogenous leukemia (CML), Hodgkin's lymphoma, non-Hodgkin's lymphoma, B-cell lymphoma, follicular lymphoma, cutaneous T-cell lymphoma), pancreatic cancer, bladder cancer, squamous cell carcinoma of the head and neck (H&NSCC also called SCCHN), genitourinary cancer, advanced cutaneous squamous cell carcinoma, liver metastases, mesothelioma, gastroesophageal cancer, Merkel cell carcinoma, and urothelial carcinoma.

59. A method of treating cancer in a subject, the method comprising:
a) administering to a subject having cancer an effective amount of a primary cell-derived biologic comprising IL-1β, IL-2, IL-6, IL-8, TNF-α, and IFN-α; and
b) administering to the subject an effective amount of an antagonist of cytotoxic T-lymphocyte-associated protein 4 (CTLA-4), wherein the administration of the primary cell-derived biologic and the administration of the antagonist occur at different locations in the subject and/or at different times.

60. The method of claim 59, wherein at least one administration of the primary cell-derived biologic occurs prior to at least one administration of the antagonist.

61. The method of claim 60, wherein at least one administration of the primary cell-derived biologic occurs prior to at least one administration of the antagonist and at least one further administration of the primary cell-derived biologic occurs after the at least one administration of the antagonist.

62. The method of claim 61, wherein at least one administration of the antagonist occurs prior to at least one administration of the primary cell-derived biologic.

63. The method of claim 62, wherein at least one administration of the antagonist occurs prior to at least one administration of the primary cell-derived biologic and at least one further administration of the antagonist occurs after the at least one administration of the primary cell-derived biologic.

64. The method of any one of claims 59 to 63, wherein the primary cell-derived biologic is administered subcutaneously or perilymphatically and the antagonist is administered intravenously.

65. The method of any one of claims 59 to 64, wherein the primary cell-derived biologic is administered once a day up to 10 days and the antagonist is administered once every three to twelve weeks.

66. The method of any one of claims 59 to 65, wherein the antagonist is an antisense oligonucleotide, a short interfering RNA (siRNA), small molecule, a peptide, or an antibody.

67. The method of claim 66, wherein the antagonist is an antibody.

68. The method of claim 67, wherein the antibody is a human or humanized antibody.

69. The method of claim 68, wherein the antibody is selected from the group consisting of ipilimumab and tremelimumab.

70. The method of any one of claims 69 to 69, wherein the subject is refractory to treatment with the antagonist prior to administration of the primary cell-derived biologic.

71. The method of any one of claims 59 to 70, wherein a level of CTLA-4 in a tumor of the subject increases after administration of the primary cell-derived biologic.

72. A method of treating cancer in a subject, the method comprising:
a) administering to a subject having cancer an effective amount of a primary cell-derived biologic comprising IL-1β, IL-2, IL-6, IL-8, TNF-α, and IFN-γ; and
b) administering to the subject an effective amount of an antagonist of cytotoxic T-lymphocyte-associated protein 4 (CTLA-4), wherein the antagonist is selected from the group consisting of ipilimumab and tremelimumab.

73. The method of any one of claims 59 to 72, wherein the effective amount of the primary cell-derived biologic administered to the subject includes a ratio of IL-1β International Units (IU) to IL-2 IU of 0.45 to 1.37, a ratio of IFN-γ IU to IL-2 IU of 0.19 to 0.39, a ratio of TNF-α IU to IL-2 IU of 0.53 to 1.26, a ratio of IL-6 IU to IL-2 IU of 1.16 to 6.06, and a ratio of IL-8 IU to IL-2 IU of 0.15 to 0.51.

74. The method of any one of claims 59 to 73, wherein the effective amount of the primary cell-derived biologic administered to the subject includes at least 1 IU of each of IL-1β, IL-2, IL-6, IL-8, and TNF-α.

75. The method of any one of claims 59 to 74, wherein the effective amount of the primary cell-derived biologic administered to the subject includes 22-657 International Units (IU) or 220-6,700 pcg of IL-1β, 29-478 IU or 1730-28,100 pcg of IL-2, 10-185 IU or 560-10,900 pcg of IFN-γ, 29-600 IU or 580-12,000 pcg of TNF-α, 34-2,895 IU or 260-22,100 pcg of IL-6 and 5-244 IU or 4,610-243,600 of IL-8.

76. The method of any one of claims 59 to 75, wherein the primary cell-derived biologic further comprises GM-CSF and G-CSF.

77. The method of any one of claims 59 to 76, further comprising administering to the subject a chemical inhibitor selected from the group consisting of alkylating agents, antimetabolites, antibiotics, and immunomodulating agents.

78. The method of claim 77, wherein the alkylating agent is cyclophosphamide.

79. The method of any one of claims 59 to 78, further comprising administering to the subject an NSAID selected from the group consisting of indomethacin, ibuprofen, celecoxib, rofecoxib, and combinations thereof.

80. The method of claim 79, wherein the NSAID is indomethacin.

81. The method of any one of claims 59 to 80, further comprising administering zinc to the subject.

82. The method of any one of claims 59 to 81, further comprising administering an effective amount of a PD-1 or PD-L1 antagonist.

83. The method of claim 82, wherein the PD-1 or PD-L1 antagonist is an antisense oligonucleotide, a short interfering RNA (siRNA), small molecule, a peptide, or an antibody.

84. The method of claim 83, wherein the PD-1 or PD-L1 antagonist is an antibody.

85. The method of claim 84, wherein the antibody is a human or humanized antibody.

86. The method of claim 83, wherein the PD-1 or PD-L1 antagonist is selected from the group consisting of nivolumab, pidilizumab, pembrolizumab, MEDI-0680, REGN2810, AMP-224, atezolizumab, durvalumab, BMS-936559, avelumab, and CA-170.

87. A method of selecting a subject for treatment, the method comprising:
a) determining a level of CTLA-4 in a tumor sample obtained from a subject having cancer and to whom has been administered a primary cell-derived biologic comprising IL-1β, IL-2, IL-6, IL-8, TNF-α, and IFN-γ; and
b) administering to the subject an effective amount of an antagonist of CTLA-4 if the level of CTLA-4 in the tumor sample is higher than a threshold level of CTLA-4.

88. The method of claim 87, wherein determining comprises performing an assay to detect the level of CTLA-4.

89. The method of claim 88, wherein the assay is selected from the group consisting of in situ hybridization, RT-qPCR, microarray analysis, multiplexed RNA expression analysis, RNA-seq, an immunohistochemistry assay, flow cytometry, a multiplexed protein assay and a Western blot assay.

90. The method of any one of claims 87 to 89, further comprising administering the primary cell-derived biologic to the subject prior to the determining step.

91. The method of any one of claims 87 to 89, wherein the antagonist is an antisense oligonucleotide, a short interfering RNA (siRNA), small molecule, a peptide, or an antibody.

92. The method of claim 91, wherein the antagonist is an antibody.

93. The method of claim 92, wherein the antibody is a human or humanized antibody.

94. The method of claim 93, wherein the antibody is selected from the group consisting of ipilimumab and tremelimumab.

95. The method of any one of claims 87 to 94, wherein the effective amount of the primary cell-derived biologic administered to the subject includes a ratio of IL-1β International Units (IU) to IL-2 IU of 0.45 to 1.37, a ratio of IFN-γ IU to IL-2 IU of 0.19 to 0.39, a ratio of TNF-α IU to IL-2 IU of 0.53 to 1.26, a ratio of IL-6 IU to IL-2 IU of 1.16 to 6.06, and a ratio of IL-8 IU to IL-2 IU of 0.15 to 0.51.

96. The method of any one of claims 87 to 95, wherein the effective amount of the primary cell-derived biologic administered to the subject includes at least 1 IU of each of IL-1β, IL-2, IL-6, IL-8, and TNF-α.

97. The method of any one of claims 87 to 96, wherein the primary cell-derived biologic includes 22-657 International Units (IU) or 220-6,700 pcg of IL-1(3, 29-478 IU or 1730-28,100 pcg of IL-2, 10-185 IU or 560-10,900 pcg of IFN-γ, 29-600 IU or 580-12,000 pcg of TNF-α, 34-2,895 IU or 260-22,100 pcg of IL-6 and 5-244 IU or 4,610-243,600 of IL-8.

98. The method of any one of claims 87 to 97, wherein the primary cell-derived biologic further comprises GM-CSF and G-CSF.

99. A method of assessing the likelihood that a subject will be responsive to an antagonist of CTLA-4, the method comprising:
a) administering a primary cell-derived biologic comprising IL-1β, IL-2, IL-6, IL-8, TNF-α, and IFN-γ to a subject having a cancer that expresses a first level of CTLA-4 that is below a threshold level of CTLA-4; and
b) determining a second level of CTLA-4 in a tumor sample from the subject after administration of the primary cell-derived biologic, wherein a second level of CTLA-4 that is above the threshold level of CTLA-4 is indicative that the subject will be responsive to the antagonist of CTLA-4.

100. The method of claim 99, wherein determining comprises performing an assay to detect the second level of CTLA-4.

101. The method of claim 100, wherein the assay is selected from the group consisting of in situ hybridization, RT-qPCR, microarray analysis, multiplexed RNA expression analysis, RNA-seq, an immunohistochemistry assay, flow cytometry, a multiplexed protein assay and a Western blot assay.

102. The method of any one of claims 59 to 101, wherein the cancer is selected from the group consisting of melanoma, lung cancer (such as non-small-cell lung cancer (NSCLC)), renal cell carcinoma (RCC), prostate cancer, ovarian cancer, colorectal cancer (CRC), kidney cancer, gastric cancer, breast cancer, diffuse large B cell lymphoma (DLBCL), hematological malignancies (e.g., acute myelogenous leukemia (AML), multiple myeloma (MM), chronic lymphocytic leukemia (CLL), Chronic myelogenous leukemia (CML), Hodgkin's lymphoma, non-Hodgkin's lymphoma, B-cell lymphoma, follicular lymphoma, cutaneous T-cell lymphoma), pancreatic cancer, bladder cancer, squamous cell carcinoma of the head and neck, genitourinary cancer, advanced cutaneous squamous cell carcinoma, liver metastases, mesothelioma, gastroesophageal cancer, Merkel cell carcinoma, and urothelial carcinoma.


SEQUENCE LISTING (6 sequences)

SEQ ID NO: 1 - protein, 153 aa, Homo sapiens
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 2 - protein, 144 aa, Homo sapiens
LSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIIS

SEQ ID NO: 3 - protein, 183 aa, Homo sapiens
VPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 4 - protein, 79 aa, Homo sapiens
EGAVLPRSAKELRCQCIKTYSKPFHPKFIKELRVIESGPHCANTEIIVKLSDGRELCLDPKENWVQRVVEKFLKRAENS

SEQ ID NO: 5 - protein, 138 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRG

SEQ ID NO: 6 - protein, 144 aa, Homo sapiens
PVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGII